(19) United States
(12) Patent Application Publication
(10) Pub. No.: US 2026/0085055 A1
(43) Pub. Date: Mar. 26, 2026

(54) NOVEL MONOMER AND CHEMICALLY RECYCLABLE POLYMER MATERIAL COMPRISING THE SAME

(71) Applicant: KOREA INSTITUTE OF SCIENCE AND TECHNOLOGY, Seoul (KR)

(72) Inventors: Tae Ann KIM, Seoul (KR); Ju Ho LEE, Seoul (KR); Jae Hong KIM, Seoul (KR); Jung Hwan BYUN, Seoul (KR); Kyung June CHO, Seoul (KR)

(73) Assignee: KOREA INSTITUTE OF SCIENCE AND TECHNOLOGY, Seoul (KR)

(21) Appl. No.: 19/307,755

(22) Filed: Aug. 22, 2025

Related U.S. Application Data
(63) Continuation of application No. PCT/KR2024/015971, filed on Oct. 21, 2024.

(30) Foreign Application Priority Data
Sep. 20, 2024 (KR) 10-2024-0127659

(51) Int. Cl. C07D 311/16; C07C 43/235; C07C 65/21; C07C 69/74; C07C 69/76; C07C 211/45; C07C 233/58; C07C 251/24; C07C 275/28; C07F 5/04; C08F 134/02

(52) U.S. Cl. CPC C07D 311/16; C07C 43/235; C07C 65/21; C07C 69/74; C07C 69/76; C07C 211/45; C07C 233/58; C07C 251/24; C07C 275/28; C07F 5/04; C08F 134/02

(57) ABSTRACT

The present invention provides a novel monomer, which provides a new functionality by including a novel monomer formed by a bond between a pentagonal cyclic olefin molecule and a photosensitive molecule, may control mechanical and thermal properties, and is formed by a bond between Chemical Formula 1 and any one of Chemical Formulae 2 to 4 in order to perform chemical recycling through reversible chemical changes according to the external stimulus.

### CROSS-REFERENCE TO RELATED APPLICATIONS

This application is a continuation-in-part of International Application No. PCT/KR2024/015971 filed on Oct. 21, 2024, which claims the benefit of Korean Patent Application No. 10-2024-0127659 filed on Sep. 20, 2024, in the Korean Intellectual Property Office, the entire disclosures of which are incorporated herein by reference for all purposes.

### TECHNICAL FIELD

The present invention relates to a novel monomer and a polymer material including the same, the polymer material having adjustable properties and being chemically recyclable.

### BACKGROUND ART

A polymer material is a substance made up of very large molecular chains, and has a structure in which monomers, which are several small molecules, are repeatedly linked together. The types of structures in which polymer materials are repeatedly linked include a linear structure in which the polymer materials are linked in a row, a branched structure in which other chains branch off from the main chain like a branch, and a network structure which is in a form in which polymers cross each other in various directions and are linked like a network.

Among them, a polymer material having damage sensing and self-healing functions is a special type of polymer capable of restoring itself in response to physical damage, thermal stress or chemical stress applied from the outside. Since these polymer materials can automatically repair damaged areas, the lifespan of the material may be extended and maintenance and repair costs may be reduced. Polymer materials having damage sensing and self-healing functions are of great interest in fields such as aerospace, automotive and electronics.

However, polymer materials having damage sensing and self-healing functions are frequently exposed to external impact or damage, and due to their characteristics that the degree of impact or damage varies, the polymer materials may lead to an increase in waste plastic because it is difficult to use the polymer materials semi-permanently. Polymer materials having damage sensing and self-healing functions, which have been studied in the related art, are prepared by a method of injecting substances capable of expressing functionality in the form of capsules or channels, but in this case, the physical properties of the material itself may deteriorate significantly, and it is difficult to respond to repeated damage to the same site. The prior art document US 2023-0365743 A1 discloses a fused ring monomer as a chemically recyclable polymer, but does not disclose a new monomer having damage sensing and self-healing functions.

Recently, there have been cases where polymer materials having damage sensing and self-healing functions are implemented using dynamic covalent bonds, but due to the characteristics that different functional molecules need to be introduced, there is a problem in that the polymer materials need to be subjected to complicated synthesis and preparation methods. Therefore, there is a highlighted need for a new polymer material that can be used semi-permanently and has damage sensing and self-healing functions through chemical recycling without complicated synthesis and preparation methods.

### DISCLOSURE

### Technical Problem

An object of the present invention is to provide a novel monomer formed by bonding a pentagonal cyclic olefin molecule and a photosensitive molecule, and a polymer material having a new functionality, which includes the novel monomer.

Another object of the present invention is to provide a polymer material including a novel monomer capable of controlling mechanical and thermal properties.

Still another object of the present invention is to provide a polymer material including a novel monomer that is chemically recyclable through a reversible chemical change according to an external stimulus.

### Technical Solution

To achieve the above-described objects, there is provided a novel monomer formed by a bond between the following Chemical Formula 1 and any one of the following Chemical Formulae 2 to 4.

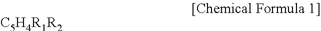

Here, Chemical Formula 1 is a ring type, R₁ is a hydroxyl group, a carboxyl group, an amine group, chlorine (Cl), bromine (Br) or iodine (I), and R₂ is a hydroxyl group or hydrogen.

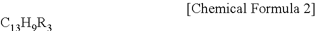

Here, R₃ is a carboxyl group, an isocyanate group, a halogen group, a halide methyl group, an amine group, a hydroxymethyl group, a boronic acid group, an aldehyde group, a phenylmethanimine group, or an ethylene group, and the halogen element is chlorine, bromine, or iodine.

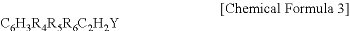

Here, R₄ is hydrogen, a methoxy group, an acetoxy group, a trifluoromethyl group, a methylseleno group, a hydroxyl group, bromine, chlorine or an amine group, R₅ is a hydroxyl group or hydrogen, R₆ is a methoxy group or hydrogen, and Y is a carboxyl group, an acyl chloride group, or chlorine.

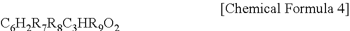

Here, R₇ is a hydroxyl group, an amine group, bromine, or chlorine, R₈ is hydrogen, a methoxy group, an acetyl group, a methyl group, a hydroxyl group, or chlorine, and R₉ is a trifluoromethyl group, a methyl group, or hydrogen.

### Advantageous Effects

The effects of the present invention obtained through the above-described means for solution are as follows.

The novel monomer provided by the present invention is in a form in which a pentagonal cyclic olefin molecule is bonded to a photosensitive molecule having fluorescent characteristics, and can provide a novel monomer having a new functionality different from the physical properties of the pentagonal cyclic olefin molecule.

Further, the polymer material provided by the present invention can be copolymerized by adjusting the contents of the novel monomer, which is in the form of a bond between a pentagonal cyclic olefin molecule and a photosensitive molecule, and a cyclopentene derivative, thereby easily controlling the mechanical and thermal properties.

In addition, the polymer material provided by the present invention can be reversibly depolymerized and restored to its original molecular structure through an external stimulus such as heat or mechanical stress, and the polymer material has damage sensing and self-healing functions, and thus can be chemically recycled.

### DESCRIPTION OF DRAWINGS

FIGS. 1A and 1B are conceptual views illustrating a structure of cyclopentene and anthracene bonded by a substituent, which is one embodiment of the novel monomer provided by the present invention, and polymerization and depolymerization under specific conditions.

FIG. 2 is a conceptual view illustrating the formation of the polymer material, the depolymerization of the polymer material, and the characteristics of the polymer material, provided by the present invention.

FIG. 3 is a view illustrating a process of preparing the polymer material according to one embodiment provided by the present invention in the form of a film.

FIG. 4 is a view illustrating a process in which the polymer material prepared according to the embodiment is formed as a chemically crosslinked polymer film by ultraviolet light transmission.

FIGS. 5A to 5C are graphs illustrating the changes in heat flow, storage modulus, and tan δ according to the temperature from the difference in copolymerization ratio of the polymer material prepared according to the embodiment.

FIGS. 6A and 6B are graphs illustrating the ultraviolet absorption and fluorescent characteristics according to wavelengths from the difference in copolymerization ratio of the polymer material prepared according to the embodiment.

FIGS. 7A to 7C are graphs illustrating the changes in storage modulus and tan δ according to the temperature from the difference in copolymerization ratio of chemically crosslinked polymer networks composed of the polymer material prepared according to the embodiment.

FIG. 8 is a graph for confirming the photodimerization efficiency of the chemically crosslinked polymer network prepared according to the embodiment.

FIG. 9 is a graph for confirming the photodimerization efficiency of chemically crosslinked polymer networks having different copolymerization ratios, prepared according to the embodiment.

FIGS. 10A to 10C are graphs for grasping hardness and mechanical properties of polymer materials and chemically crosslinked polymer networks having different copolymerization ratios, prepared according to the embodiment.

FIGS. 11A and 11B are photographs illustrating the self-healing ability of polymer materials having different copolymerization ratios prepared according to the embodiment according to the external stimulus.

FIGS. 12A and 12B are views illustrating the dual-shape memory properties of the polymer material prepared according to the embodiment.

FIG. 13 is a view illustrating a process of recycling the polymer material prepared according to the embodiment through depolymerization.

### MODE FOR DISCLOSURE

Hereinafter, the novel monomer and the chemically recyclable polymer material including the same related to the present invention will be described in more detail with reference to drawings.

In the present specification, like reference numbers are used to designate like constituents even though they are in different Examples, and the description thereof will be omitted.

When it is determined that the detailed description of the publicly known art related in describing the Examples disclosed in the present specification may obscure the gist of the Examples disclosed in the present specification, the detailed description thereof will be omitted.

The accompanying drawings are provided to easily understand the Examples disclosed in the present specification, and it is to be appreciated that the technical spirit disclosed in the present specification is not limited by the accompanying drawings, and the accompanying drawings include all the changes, equivalents, and substitutions included in the spirit and the technical scope of the present invention.

In the following description, singular expressions include plural expressions unless the context clearly indicates otherwise.

In the present application, the term “include” or “have” is intended to indicate the presence of a characteristic, number, step, operation, constituent element, part or any combination thereof described in the specification, and it should be understood that the possibility of the presence or addition of one or more other characteristics or numbers, steps, operations, constituent elements, parts or any combination thereof is not precluded.

Hereinafter, a chemically recyclable polymer material proposed by the present invention will be described.

A novel monomer may be formed by a bond between the following Chemical Formula 1 and any one of the following Chemical Formulae 2 to 4.

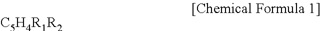

Here, Chemical Formula 1 is a ring type, R₁ is a hydroxyl group, a carboxyl group, an amine group, chlorine (CI), bromine (Br) or iodine (I), and R₂ is a hydroxyl group or hydrogen.

Chemical Formula 1 may be in a form in which cyclopentene, which is a pentagonal cyclic olefin molecule, has one or two substituents.

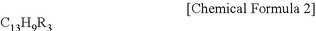

Here, R₃ is a carboxyl group, an isocyanate group, a halogen group, a halide methyl group, an amine group, a hydroxymethyl group, a boronic acid group, an aldehyde group, a phenylmethanimine group, or an ethylene group, and the halogen element is chlorine, bromine, or iodine.

Chemical Formula 2 may be in a form in which anthracene has one substituent.

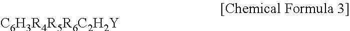

Here, R₄ is hydrogen, a methoxy group, an acetoxy group, a trifluoromethyl group, a methylseleno group, a hydroxyl group, bromine, chlorine or an amine group, R₅ is a hydroxyl group or hydrogen, R₆ is a methoxy group or hydrogen, and Y is a carboxyl group, an acyl chloride group, or chlorine.

Chemical Formula 3 may be in a form in which cinnamic acid has one to three substituents, or in a form in which the carboxyl group of cinnamic acid to which one to three substituents are attached is substituted with acyl chloride or chlorine.

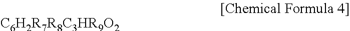

Here, R₇ is a hydroxyl group, an amine group, bromine, or chlorine, R₈ is hydrogen, a methoxy group, an acetyl group, a methyl group, a hydroxyl group, or chlorine, and R₉ is a trifluoromethyl group, a methyl group, or hydrogen.

Chemical Formula 4 may be in a form in which coumarin has 1 to 3 substituents.

Here, Chemical Formula 1 may be represented by any one of the following Chemical Formulae 1-1 to 1-5.

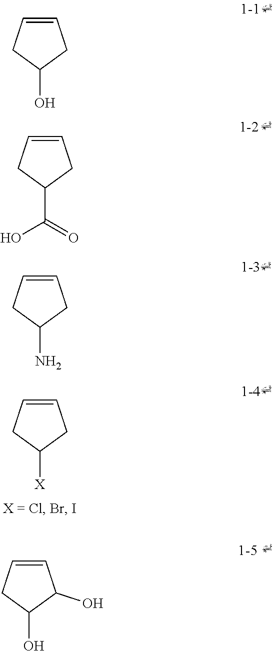

Meanwhile, Chemical Formula 2 may be represented by any one of the following Chemical Formulae 2-1 to 2-10.

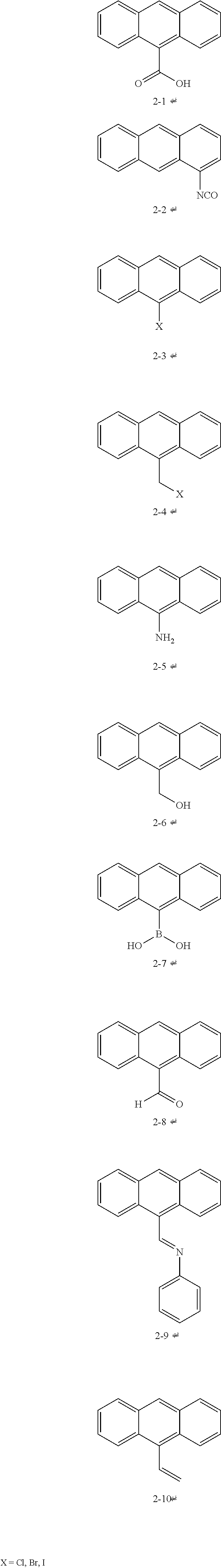

In addition, Chemical Formula 3 may be represented by any one of the following Chemical Formulae 3-1 to 3-14.

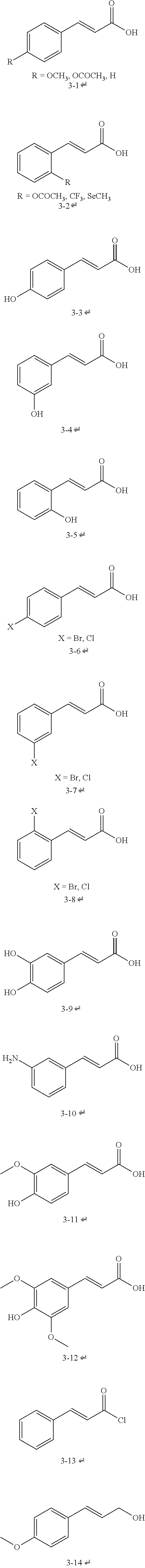

Furthermore, the following Chemical Formula 4 may be represented by any one of the following Chemical Formulae 4-1 to 4-11.

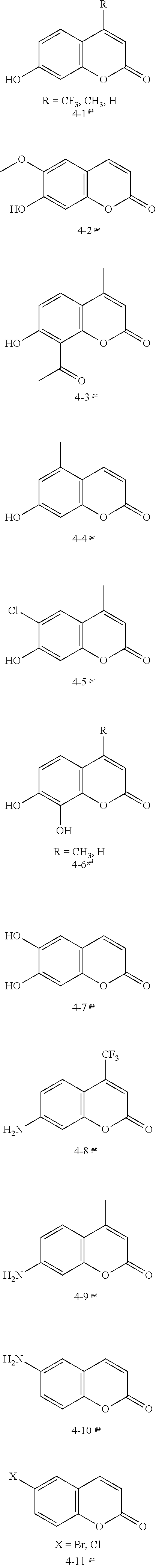

For the novel monomer provided by the present invention, a novel monomer which is any one of the following Chemical Formulae 5-1 to 5-20 may be formed by bonding any one of Chemical Formulae 1-1 to 1-5, which are in a substituent form of cyclopentene, to any one of Chemical Formulae 2-1 to 2-10, which are in a substituent form of anthracene.

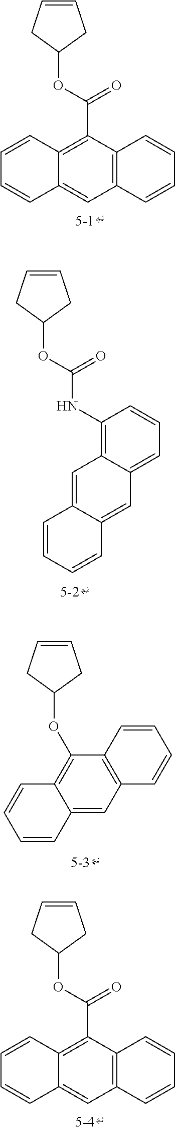 [SMILES: O=C(OC1CC=CC1)c1c2ccccc2cc2ccccc12 ; O=C(Nc1cccc2cc3ccccc3cc12)OC1CC=CC1 ; C1=CCC(Oc2c3ccccc3cc3ccccc23)C1 ; O=C(OC1CC=CC1)c1c2ccccc2cc2ccccc12]

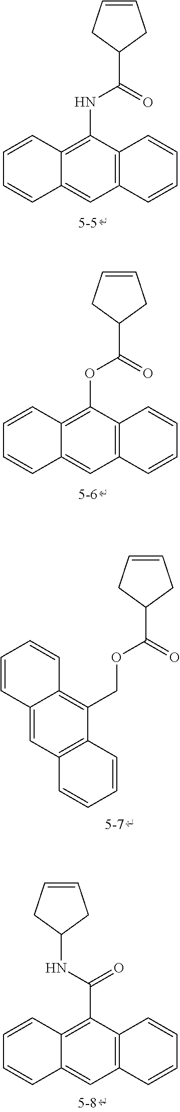 [SMILES: O=C(Nc1c2ccccc2cc2ccccc12)C1CC=CC1 ; O=C(Oc1c2ccccc2cc2ccccc12)C1CC=CC1 ; O=C(OCc1c2ccccc2cc2ccccc12)C1CC=CC1 ; O=C(NC1CC=CC1)c1c2ccccc2cc2ccccc12]

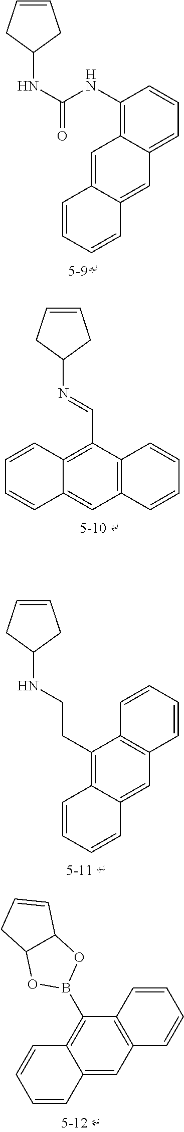 [SMILES: O=C(Nc1cccc2cc3ccccc3cc12)NC1CC=CC1 ; C1=CCC(/N=C/c2c3ccccc3cc3ccccc23)C1 ; C1=CCC(NCCc2c3ccccc3cc3ccccc23)C1 ; C1=CC2OB(c3c4ccccc4cc4ccccc34)OC2C1]

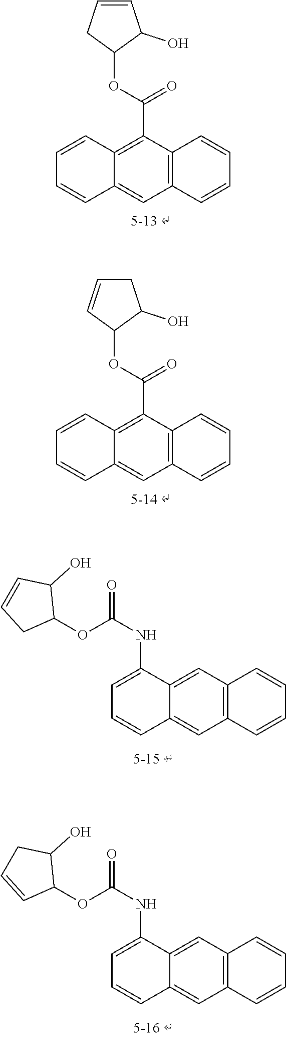 [SMILES: O=C(OC1CC=CC1O)c1c2ccccc2cc2ccccc12 ; O=C(OC1C=CCC1O)c1c2ccccc2cc2ccccc12 ; O=C(Nc1cccc2cc3ccccc3cc12)OC1CC=CC1O ; O=C(Nc1cccc2cc3ccccc3cc12)OC1C=CCC1O]

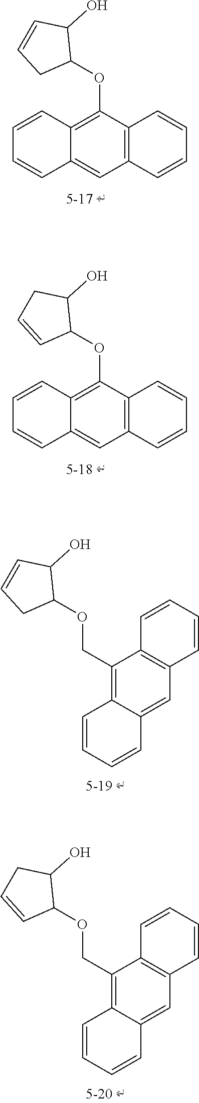 [SMILES: OC1C=CCC1Oc1c2ccccc2cc2ccccc12 ; OC1CC=CC1Oc1c2ccccc2cc2ccccc12 ; OC1C=CCC1OCc1c2ccccc2cc2ccccc12 ; OC1CC=CC1OCc1c2ccccc2cc2ccccc12]

Among the above Chemical Formula 5, Chemical Formulae 5-1 to 5-4 may include Chemical Formula 1-1 as a part of the monomer, and Chemical Formulae 5-5 to 5-8 may include Chemical Formula 1-2 as a part of the monomer. Further, Chemical Formulae 5-8 to 5-11 may include Chemical Formula 1-3 as a part of the monomer, and Chemical Formulae 5-1 and 5-4 may include Chemical Formula 1-4 as a part of the monomer. Furthermore, Chemical Formula 5-12 to 5-20 may include Chemical Formula 1-5 as a part of the monomer.

In addition, for the novel monomer provided by the present invention, a novel monomer which is any one of the following Chemical Formulae 6-1 to 6-65 may be formed by bonding any one of Chemical Formulae 1-1 to 1-5, which are in a substituent form of cyclopentene, to any one of Chemical Formulae 3-1 to 3-14, which are in a substituent form of cinnamic acid.

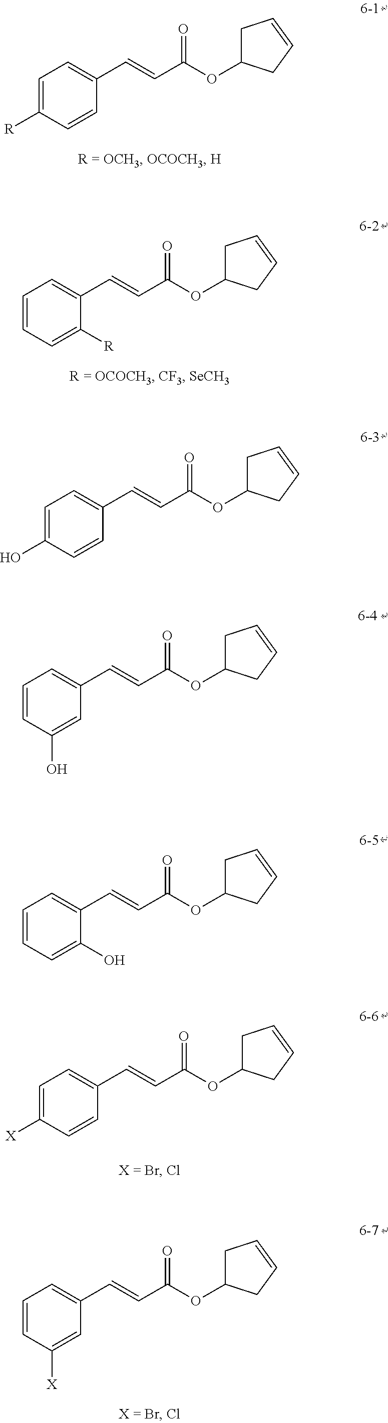

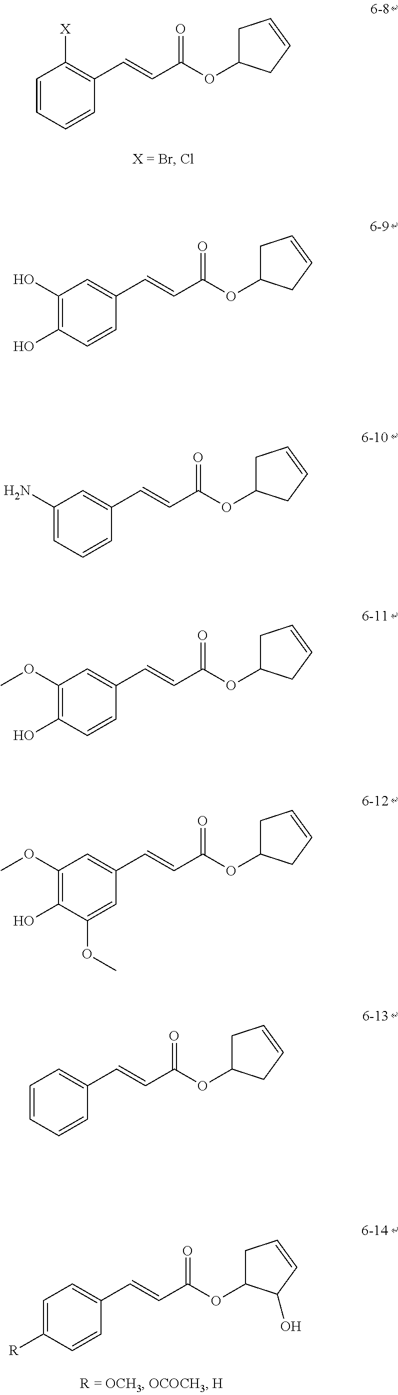

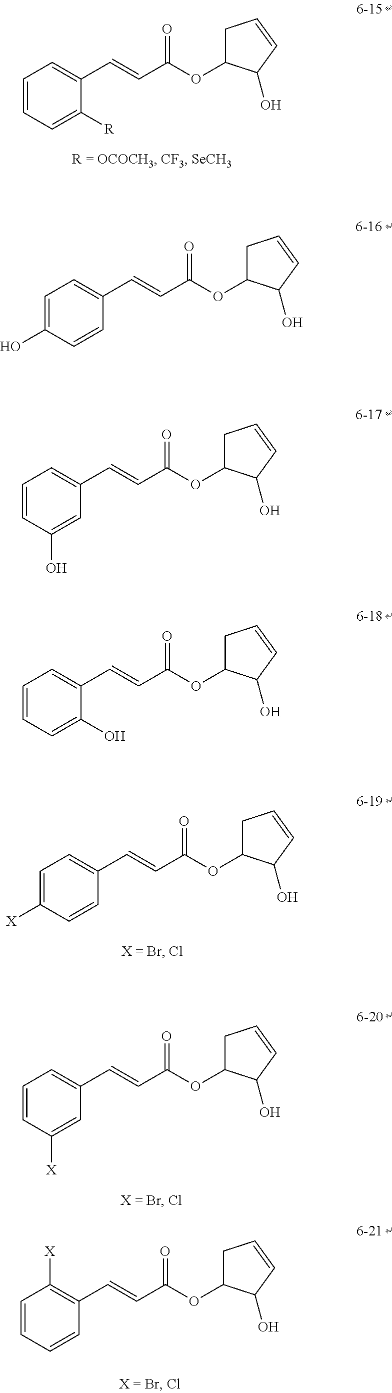

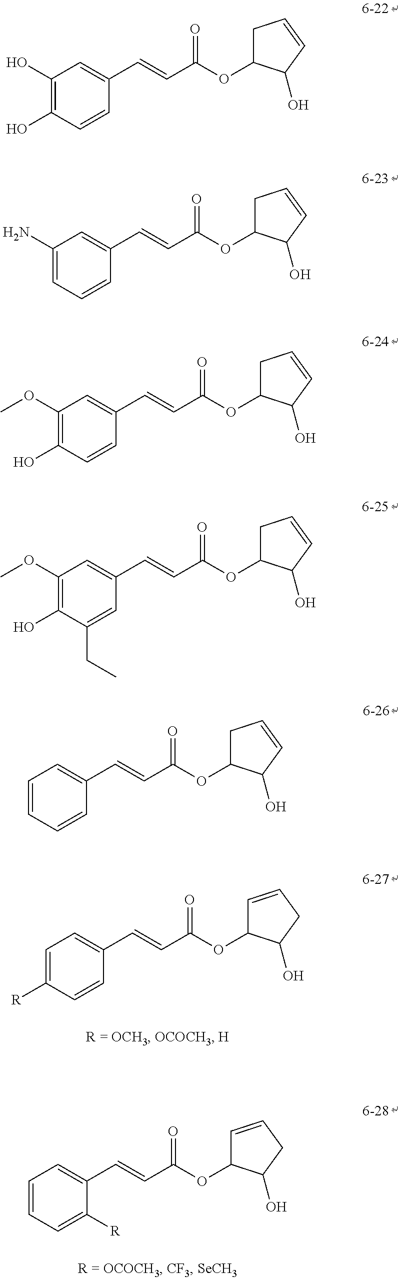

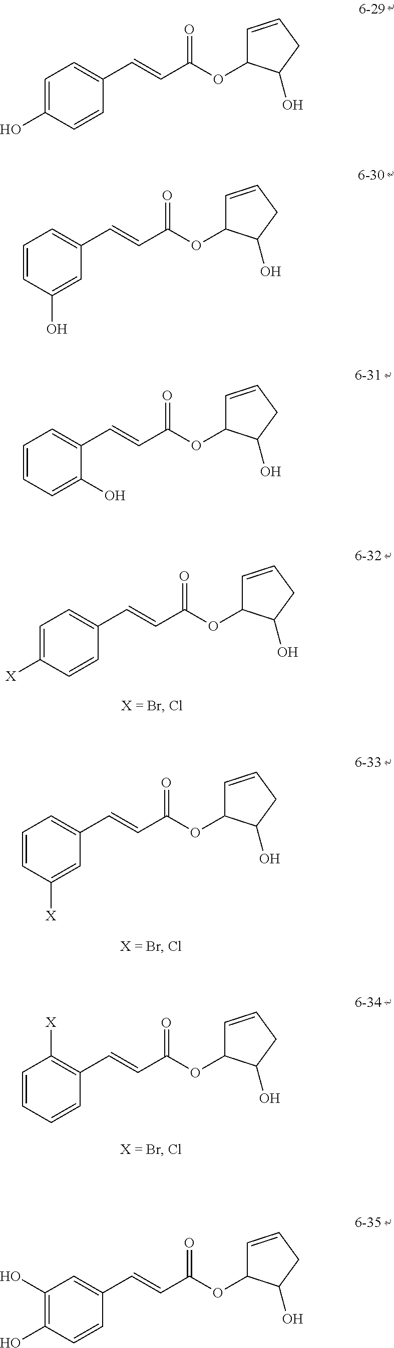

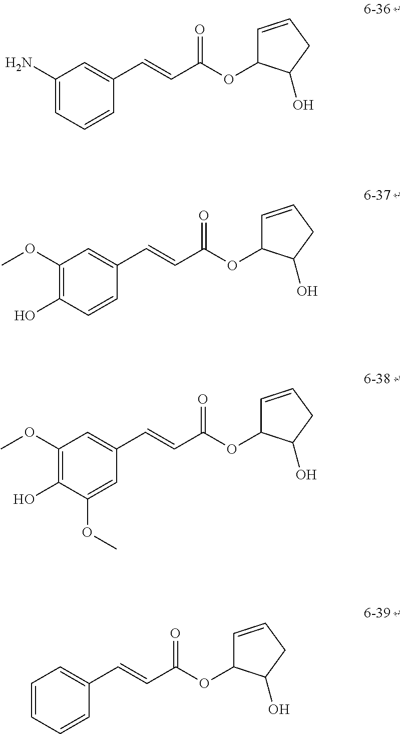 [SMILES: Nc1cccc(/C=C/C(=O)OC2C=CCC2O)c1 ; COc1cc(/C=C/C(=O)OC2C=CCC2O)ccc1O ; COc1cc(/C=C/C(=O)OC2C=CCC2O)cc(OC)c1O ; O=C(/C=C/c1ccccc1)OC1C=CCC1O]

Chemical Formulae 6-1 to 6-39 correspond to chemical formulae formed by bonding any one of Chemical Formulae 1-1 and 1-5 having a hydroxyl group (—OH) as a substituent in Chemical Formula 1 to any one of Chemical Formulae 3-1 to 3-13 having a carboxyl group (—COOH) or an acyl chloride group as a substituent in Chemical Formula 3.

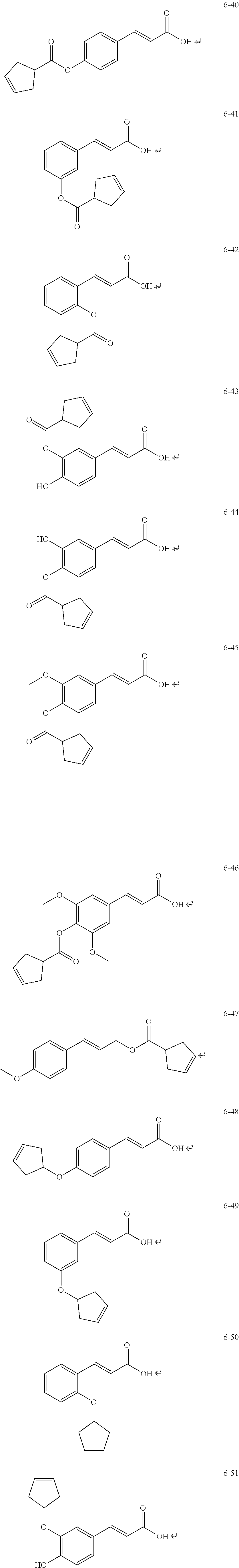

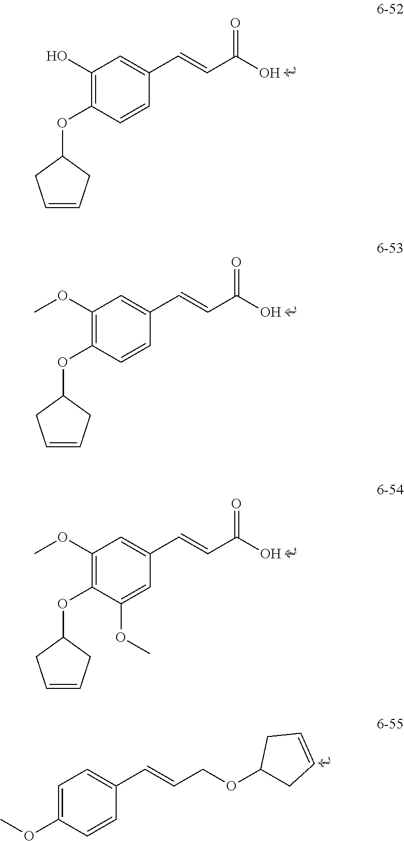 [SMILES: O=C(O)/C=C/c1ccc(OC2CC=CC2)c(O)c1 ; COc1cc(/C=C/C(=O)O)ccc1OC1CC=CC1 ; COc1cc(/C=C/C(=O)O)cc(OC)c1OC1CC=CC1 ; COc1ccc(/C=C/COC2CC=CC2)cc1]

Chemical Formulae 6-40 to 6-55 correspond to chemical formulae formed by bonding any one of Chemical Formulae 1-2 and 1-5 having a carboxyl group (—COOH) or a halogen group as a substituent in Chemical Formula 1 to any one of Chemical Formulae 3-3, 3-4, 3-5, 3-9, 3-11, 3-12, and 3-14 having a hydroxyl group (—OH) as a substituent in Chemical Formula 3.

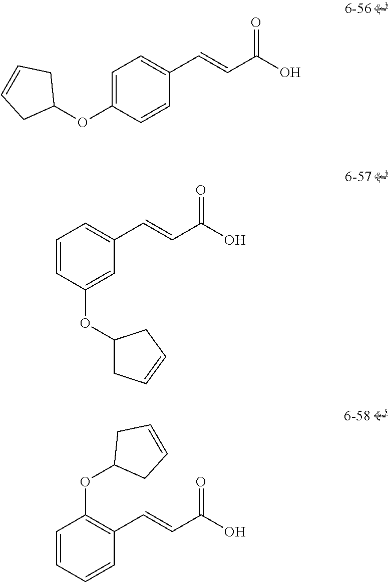

Chemical Formulae 6-56 to 6-64 correspond to chemical formulae formed by bonding any one of Chemical Formulae 1-1 and 1-5 having a hydroxyl group (—OH) as a substituent in Chemical Formula 1 to any one of Chemical Formulae 3-6 to 3-8 having

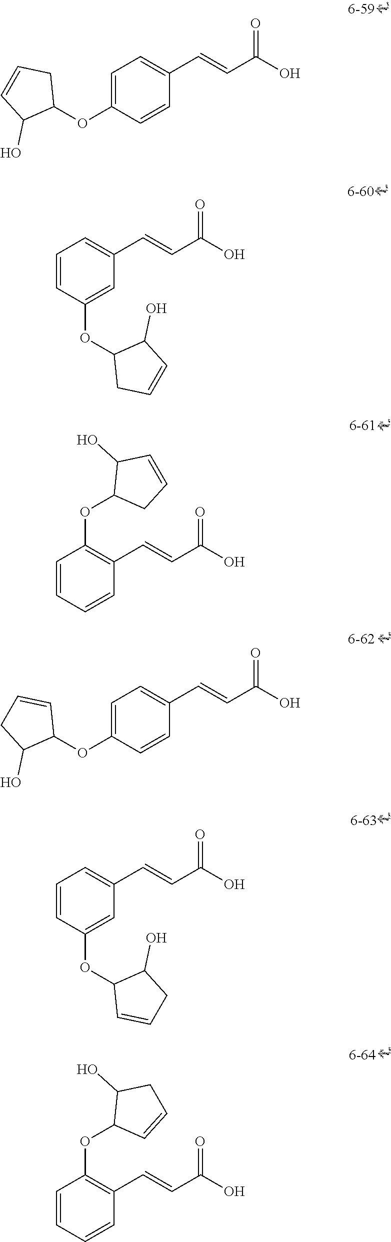 [SMILES: O=C(O)/C=C/c1ccc(OC2CC=CC2O)cc1 ; O=C(O)/C=C/c1cccc(OC2CC=CC2O)c1 ; O=C(O)/C=C/c1ccccc1OC1CC=CC1O ; O=C(O)/C=C/c1ccc(OC2C=CCC2O)cc1 ; O=C(O)/C=C/c1cccc(OC2C=CCC2O)c1 ; O=C(O)/C=C/c1ccccc1OC1C=CCC1O]

bromine or chlorine of a halogen group as a substituent in Chemical Formula 3.

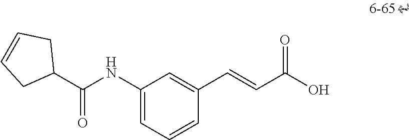 [SMILES: O=C(O)/C=C/c1cccc(NC(=O)C2CC=CC2)c1]

Chemical Formula 6-65 corresponds to a chemical formula formed by bonding Chemical Formula 1-2 having a carboxyl group (—COOH) as a substituent in Chemical Formula 1 to Chemical Formula 3-10 having an amine group (—NH₂) as a substituent in Chemical Formula 3.

Furthermore, for the novel monomer provided by the present invention, a novel monomer which is any one of the following Chemical Formulae 7-1 to 7-27 may be formed by bonding any one of Chemical Formulae 1-1 to 1-5, which are in a substituent form of cyclopentene, to any one of Chemical Formulae 4-1 to 4-11, which are in a substituent form of coumarin.

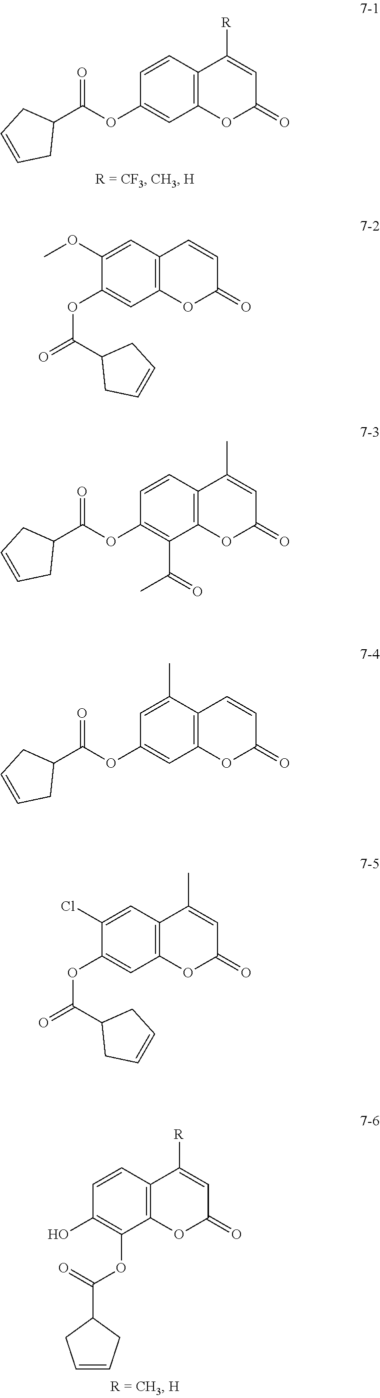

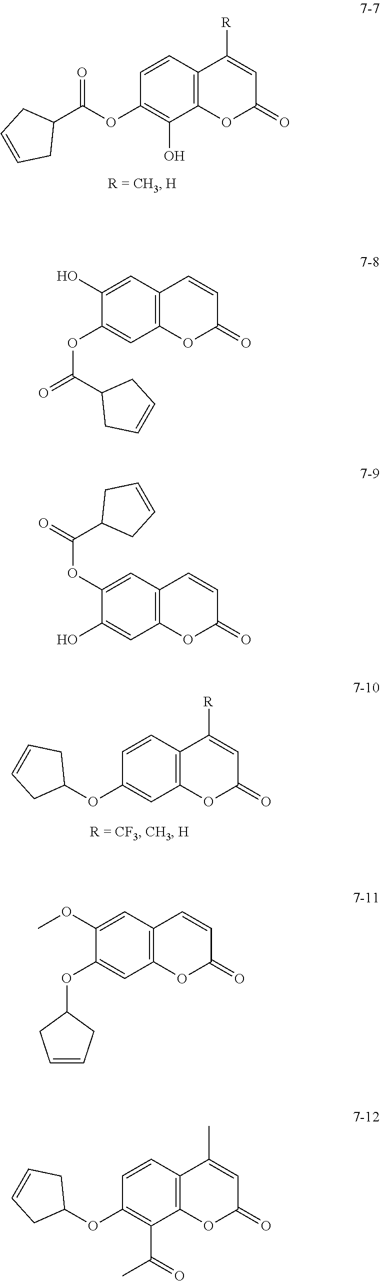

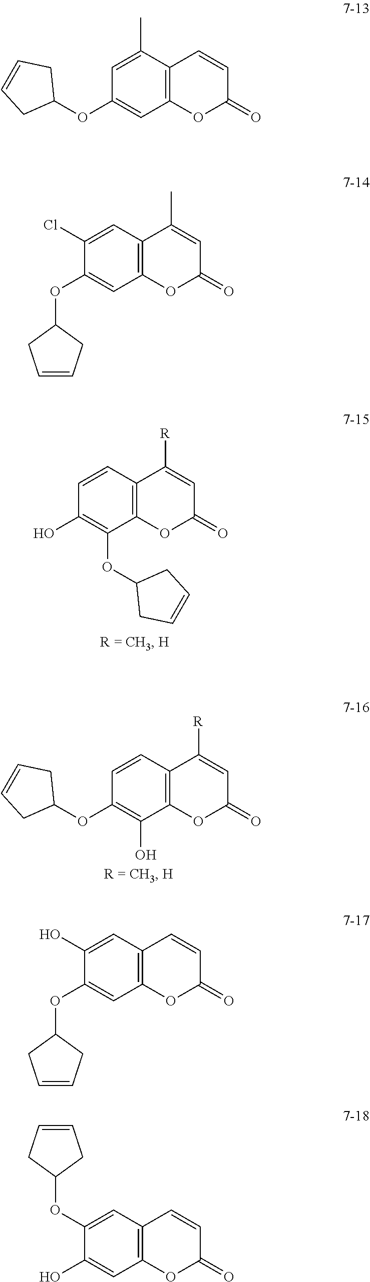

Chemical Formulae 7-1 to 7-18 correspond to chemical formulae formed by bonding any one of Chemical Formulae 1-2 and 1-4 having a carboxyl group (—COOH) or a halogen group as a substituent in Chemical Formula 1 to any one of Chemical Formulae 5-1 to 5-7 having a hydroxyl group (—OH) as a substituent in Chemical Formula 5.

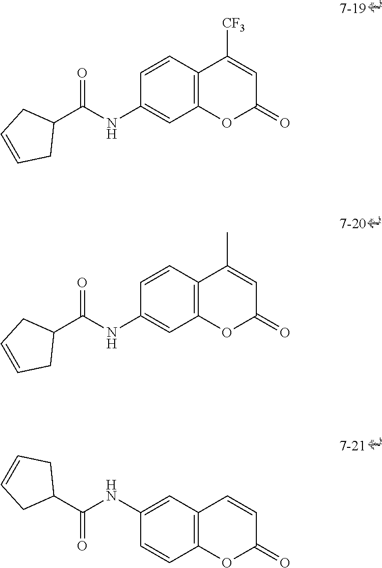 [SMILES: O=C(Nc1ccc2c(C(F)(F)F)cc(=O)oc2c1)C1CC=CC1 ; Cc1cc(=O)oc2cc(NC(=O)C3CC=CC3)ccc12 ; O=C(Nc1ccc2oc(=O)ccc2c1)C1CC=CC1]

Chemical Formulae 7-19 to 7-21 correspond to chemical formulae formed by bonding Chemical Formula 1-2 having a carboxyl group (—COOH) as a substituent in Chemical Formula 1 to any one of Chemical Formulae 5-8 to 5-10 having an amine group (—NH₂) as a substituent in Chemical Formula 5.

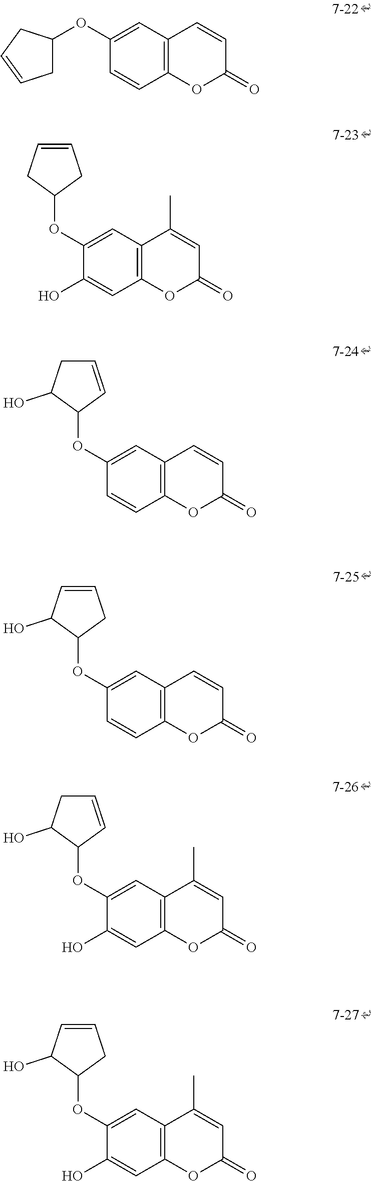 [SMILES: O=c1ccc2cc(OC3CC=CC3)ccc2o1 ; Cc1cc(=O)oc2cc(O)c(OC3CC=CC3)cc12 ; O=c1ccc2cc(OC3C=CCC3O)ccc2o1 ; O=c1ccc2cc(OC3CC=CC3O)ccc2o1 ; Cc1cc(=O)oc2cc(O)c(OC3C=CCC3O)cc12 ; Cc1cc(=O)oc2cc(O)c(OC3CC=CC3O)cc12]

Chemical Formulae 7-22 to 7-27 correspond to chemical formulae formed by bonding any one of Chemical Formulae 1-1 and 1-5 having a hydroxyl group (—OH) as a substituent in Chemical Formula 1 to any one of Chemical Formulae 5-5 and 5-11 having bromine or chlorine of a halogen group as a substituent in Chemical Formula 5.

The compound represented by Chemical Formula 5-7, formed by the combination of Chemical Formulas 1-2 and 2-6, is 9-Anthracenylmethyl 3-cyclopentene-1-cyclopentene (Anth-CP), and it can be synthesized by the following method. Here, Chemical Formula 1-2 corresponds to 9-Anthracenemethanol, and Chemical Formula 2-6 corresponds to 3-Cyclopentene-1-carboxylic acid.

9-Antracenemethanol (9-AM) (3.00 g, 14.4 mmol, 1.0 equiv.), 4-Dimethylaminopyridine (4-DMAP) (0.211 g, 1.73 mmol, 0.12 equiv.), and 1-(3-Dimethylaminopropyl)-3-ethylcarbodiimide hydrochloride (EDCI) (3.31 g, 17.3 mmol, 1.2 equiv.) were added to a flask, and the reaction was conducted under an argon atmosphere. Then, 60 ml of Dichloromethane (DCM) was added to the flask, and the mixture was stirred at room temperature for about 30 minutes until the solution became homogeneous. Subsequently, 3-Cyclopentene-1-carboxylic acid (3-CA) (1.94 g, 17.3 mmol, 1.2 equiv.) was slowly added dropwise and the mixture was stirred for 24 hours. After completion of the reaction, the reaction mixture was concentrated, and the product was purified by column chromatography using a hexane-dichloromethane mixture (1:1 mass ratio) as the eluent. Finally, the purified compound was evaporated using a rotary evaporator and dried in a vacuum oven for more than 12 hours to yield 9-Anthracenylmethyl 3-cyclopentene-1-cyclopentene as a yellow solid (4.14 g, 13.7 mmol, 95% yield).

The novel monomer provided by the present invention may have a ring strain of more than 2 kcal/mol and less than 8 kcal/mol.

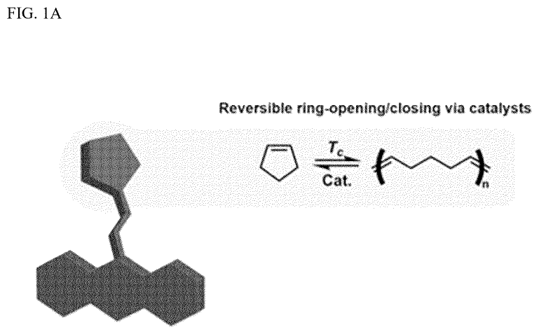

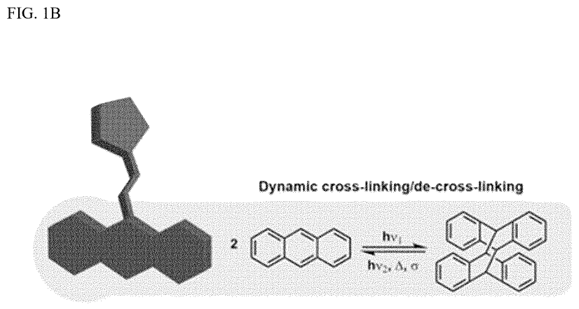

FIGS. 1A and 1B are conceptual views illustrating a structure of cyclopentene and anthracene bonded by a substituent, which is one example of the novel monomer provided by the present invention, and polymerization and depolymerization under specific conditions.

Referring to FIG. 1A, cyclopentene may be subjected to ring-opening metathesis polymerization and ring-closing metathesis depolymerization under specific catalytic conditions, and the polymerization and depolymerization of cyclopentene may be regulated by the reaction temperature. In order for a novel monomer made by bonding Chemical Formula 1 which is cyclopentene having a substituent to any one of Chemical Formulae 2 to 4 to be polymerized into a polymer material or for the polymerized polymer material to be reversibly depolymerized, the novel monomer needs to have a ring strain of more than 2 kcal/mol and less than 8 kcal/mol.

When the ring strain value is more than 8 kcal/mol, the polymer material cannot be chemically recycled because it is difficult to depolymerize the polymer material, and when the ring strain value is less than 2 kcal/mol, it is difficult to form the polymer material because the structure of a cyclopentene ring is very stable.

Referring to FIG. 1B, it can be confirmed that anthracene, which corresponds to a constituent element of one embodiment of the novel monomer provided by the present invention, is dimerized at a specific wavelength, and the dimer of anthracene returns to its original structure by mechanical force, heat, or a specific wavelength. In the polymer material, a molecule that can be attached to the substituent position of cyclopentene is a photosensitive molecule, and in order for the molecule to be attached to a substituent, the molecule in its original state before forming a crosslinking bond is a molecule having optical characteristics, and needs to possess a specific color or have fluorescent characteristics. In addition, the molecule that can be attached to the substituent position of cyclopentene needs to be a molecule that, when irradiated with light at a specific wavelength range, forms a covalent bond to be transformed into a dimer, and lose its original optical characteristics. Furthermore, the covalent bond in a molecule in the form of a dimer needs to be broken by mechanical force, allowing the molecule to exhibit optical characteristics, or the covalent bonds in the molecule needs to be broken by heat treatment or light at other wavelengths, allowing the molecule to possess self-healing functions. Chemical Formulae 2 to 4 in the present invention may satisfy the conditions for a molecule that can be attached to the substituent position of cyclopentene.

The polymer material proposed by the present invention is a chemically recyclable material and may include a novel monomer formed by a bond between Chemical Formula 1 and any one of Chemical Formulae 2 to 4.

More specifically, the polymer material proposed by the present invention may include a novel monomer formed by a bond between any one of Chemical Formulae 1-1 to 1-5 and any one of Chemical Formulae 2-1 to 2-10, Chemical Formulae 3-1 to 3-14, or Chemical Formulae 4-1 to 4-11.

In this case, the polymer material including the novel monomer may be formed by homopolymerization of the novel monomer or copolymerization between the novel monomer and a cyclopentene derivative. Here, the substituent in the cyclopentene derivative is not limited, and may be used as a derivative of cyclopentene as long as the substituent is under conditions capable of forming copolymerization between the novel monomer and the cyclopentene derivative.

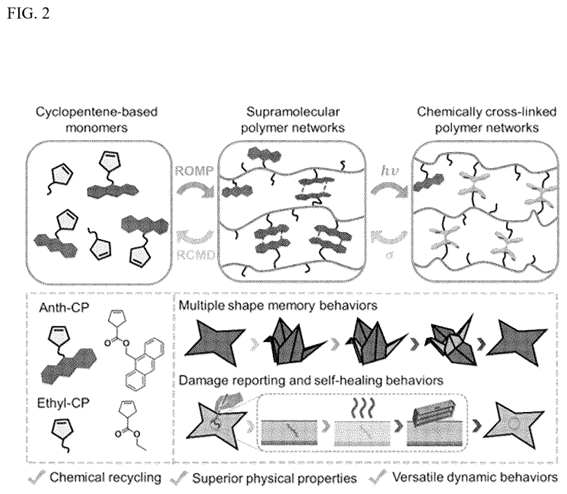

FIG. 2 is a conceptual view illustrating the formation of the polymer material, the depolymerization of the polymer material, and the characteristics of the polymer material, provided by the present invention.

Referring to FIG. 2, the polymer material provided by the present invention may form various linear polymers through copolymerization with other cyclopentene derivatives based on a novel monomer formed by a bond between a pentagonal cyclic olefin molecule and a photosensitive molecule, and the mechanical and thermal properties of the polymer material may be controlled according to the copolymerization ratio.

Here, the polymer material may form supramolecular polymer networks that have increased thermal and mechanical properties due to interactions between the photosensitive molecules located laterally. The supramolecular polymer networks may form reversible covalent bonds upon photosensitivity and be converted into chemically crosslinked polymer networks, thereby exhibiting enhanced physical properties. In addition, when a chemically crosslinked polymer network is locally irradiated with a specific wavelength, it is possible to implement shape-memory properties capable of locally fixing a shape based on the enhanced physical properties.

The crosslinked structure in the chemically crosslinked polymer network may be broken by breaking the covalent bonds by physical force, and the fluorescent characteristics of the photosensitive molecule may be again exhibited at a site exposed by the breaking of the covalent bonds, thereby showing damage sensing and self-healing characteristics. Further, the polymer material proposed by the present invention may be easily depolymerized, and thus converted into the original monomer when heated to a temperature equal to or higher than the ceiling temperature under catalytic conditions, and only a specific monomer may be recovered because the polymer material can be selectively depolymerized.

In the polymer material provided by the present invention, the molar ratio of the novel monomer in the polymer material may be 1 mol % or more. This means that the polymer material includes a novel monomer and a cyclopentene derivative, and the molar ratio of the novel monomer among them is 1 mol % or more. If the molar ratio of the novel monomer in the polymer material is less than 1 mol %, it is difficult to exhibit the damage sensing and self-healing functions of the polymer material.

Hereinafter, the novel monomer proposed by the present invention and the polymer material including the novel monomer will be described in more detail with reference to examples and drawings.

### Example 1

Anth-CP (M_{A.0}) and 8.4 mg of Grubbs 2^{nd} generation catalyst (I₀) are put into a flask, and the flask is purged with argon. Thereafter, 0.1 ml of anhydrous chloroform is injected to uniformly dissolve a monomer and a catalyst. Subsequently, Ethyl-CP (M_{E.0}) is additionally injected and the contents are stirred at a temperature between about 0° C. and 5° C. for 24 hours. Next, 0.2 ml of ethyl vinyl ether is added to deactivate the catalyst while maintaining stirring for 1 hour, and then chloroform is additionally added to dilute the reaction solution. The diluted solution is allowed to pass through a basically activated aluminum oxide column to remove the deactivated catalyst, and then the corresponding solution is condensed to a suitable volume. Thereafter, unreacted monomers are removed by precipitation in excess dimethyl ether or methanol, and a polymer finally obtained is dried under reduced pressure and then redissolved for use.

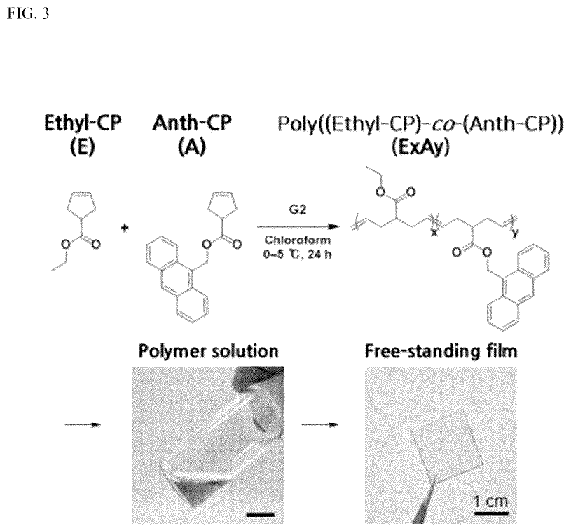

FIG. 3 is a view illustrating a process of preparing the polymer material according to one embodiment of the present invention in the form of a film.

Referring to FIG. 3, a synthesized anthracene-containing cyclopentene derivative (Anth-CP) and ethyl-3-cyclopente-1-carboxylate (Ethyl-CP) may be mixed at different ratios to prepare various copolymers in the form of ExAy, as shown in the following Table 1. When a polymer material obtained by precipitation is dissolved in a solvent, and then the solvent is slowly evaporated through a casting process, a free-standing film may be obtained by the pi-pi interactions of the anthracene molecules. Here, x and y refer to the bonding ratios of Ethyl-CP and Anth-CP.

TABLE 1

| | M_{E, O}^{a)} | M_{A, O}^{b)} | | Chloroform | Precipitation |
|---|---|---|---|---|---|
| | [mmol] | [mmol] | M_{E+A, O}/I_{O}^{c)} | (mL) | solvent |
| E1A0 | 4.75 | 0 | 480 | 0 | MeOH |
| E3A1 | 2.67 | 0.890 | 360 | 0.2 | Diethyl ether |
| E1A1 | 1.48 | 1.48 | 300 | 0.3 | Diethyl ether |
| E1A3 | 0.619 | 1.85 | 250 | 0.3 | Diethyl ether |
| E0A1 | 0 | 1.98 | 200 | 0.3 | Diethyl ether |

Here, a) and b) are the numbers of moles when Ethyl-CP (E) and Anth-CP (A) are added, respectively, at reaction time t=0, and c) represents the ratio of the number of moles of monomer to the number of moles of catalyst.

### Example 2

The polymer film formed in Example 1 is placed on a Teflon film, and then ExAys, which are polymer films, are maintained at a temperature by 30° C. higher than each of the glass transition temperatures for about 15 minutes using a heating plate. Then, the ExAys are irradiated with ultraviolet light for 15 minutes at an intensity of about 0.4 W/cm² using a UV lamp with a single long-wavelength (365 nm).

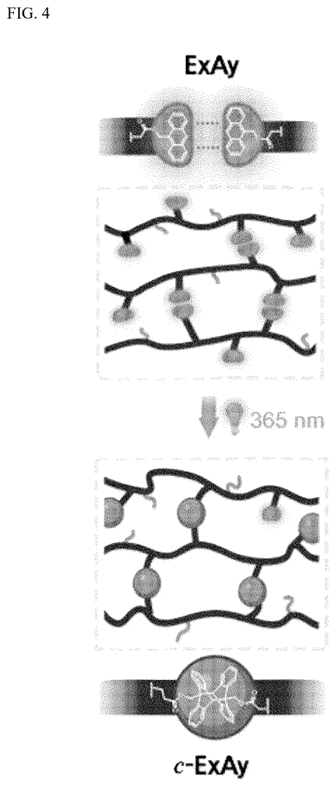

FIG. 4 is a view illustrating a process in which the polymer material prepared according to the embodiment is formed as a chemically crosslinked polymer film by ultraviolet light transmission.

Referring to FIG. 4, when a polymer film having a supramolecular polymer network structure (ExAy) is irradiated with ultraviolet light with a long wavelength longer than 300 nm under inert gas conditions, a new covalent bond is formed between two anthracene molecules, and may be transformed into a polymer film which is a chemically crosslinked polymer network (c-ExAy), and polymers with different compositions may all be prepared by the same method.

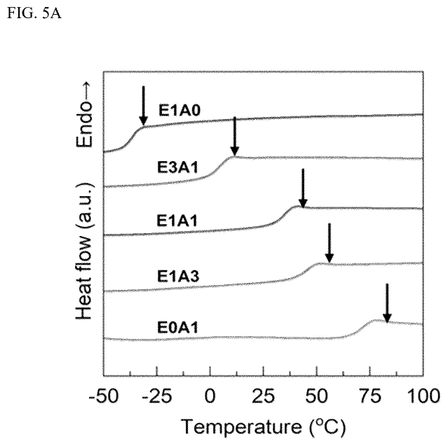

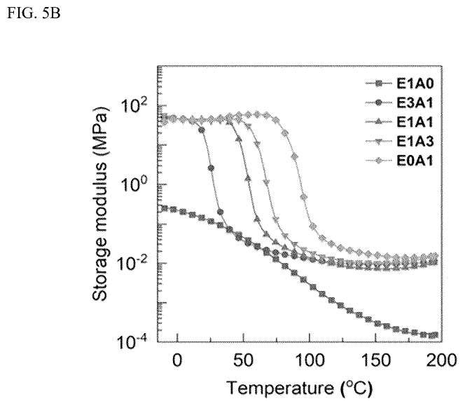

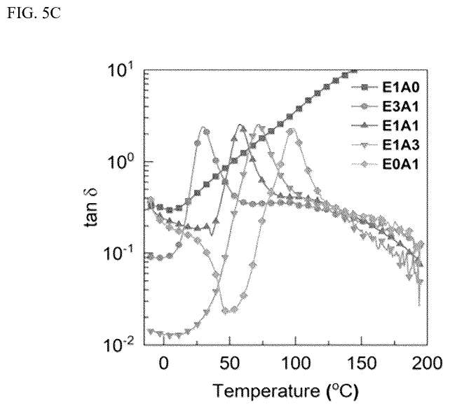

FIGS. 5A to 5C are graphs illustrating the changes in heat flow, storage modulus, and tan δ according to the temperature from the difference in copolymerization ratio of the polymer material prepared according to the embodiment.

As Ethyl-CP and Anth-CP were each mixed at different ratios, a total of five types of polymers were produced, and the values for the molecular weight and storage modulus of the monomers are shown in the following Table 2.

TABLE 2

| | Mₙ [kg mol⁻¹] | G′ at 25° C. [MPa] |
|---|---|---|
| E1A0 | 69.5 | 0.12 |
| E3A1 | 60.0 | 2.62 |
| E1A1 | 69.2 | 45.01 |
| E1A3 | 64.2 | 45.08 |
| E0A1 | 69.6 | 45.18 |

Referring to FIG. 5A, it could be confirmed that as the content of anthracene in the polymer increases, the value of heat flow decreases. Further, referring to FIG. 5B, it could be confirmed that as the content of anthracene in the polymer increases, ExAy having a high glass transition temperature is formed. In the graph of FIG. 5B, through the fact that the position having an inflection point is formed at a higher temperature, it could be seen that ExAy having a higher anthracene content has a high glass transition temperature. The reason for the high glass transition temperature is believed to be that the higher degree of crosslinking reduces the fluidity of the polymer chains.

Referring to FIG. 5C, it could be seen that the position of a peak showing the highest tan & value differs depending on the difference in content of anthracene, and it could be confirmed that the temperature value corresponding to the highest tan δ value corresponds to the glass transition temperature.

From FIGS. 5A to 5C, it could be confirmed by a rheometer that all polymers except for E1A0 formed supramolecular polymer networks due to strong interactions between anthracenes. In addition, it could be confirmed that as the content of anthracene increases, the storage modulus increases, and a similar behavior is observed in all of the rubber plateau regions after the glass transition temperature.

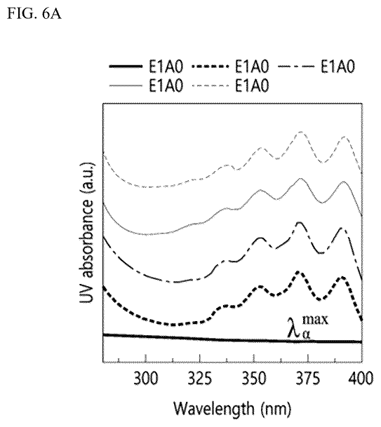

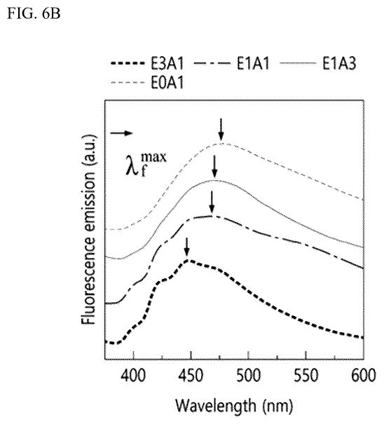

FIGS. 6A and 6B are graphs illustrating the ultraviolet absorption and fluorescent characteristics according to wavelengths from the difference in copolymerization ratio of the polymer material prepared according to the embodiment. Since the molecular structure of anthracene is maintained as it is in the supramolecular polymer network structure, it is necessary to confirm the ultraviolet absorption and fluorescent characteristics according to the anthracene content.

Referring to FIG. 6A, in all ExAys except for E1A0, a characteristic ultraviolet absorption peak of anthracene molecules appeared in the range of 330 to 400 nm, and no difference was observed according to the anthracene molecule content. In contrast, referring to FIG. 6B, in the case of fluorescent characteristics, as the content of anthracene increases, light with a gradually increasing wavelength is emitted, which is determined to be due to the redshift caused by the formation of J-aggregates between anthracene molecules.

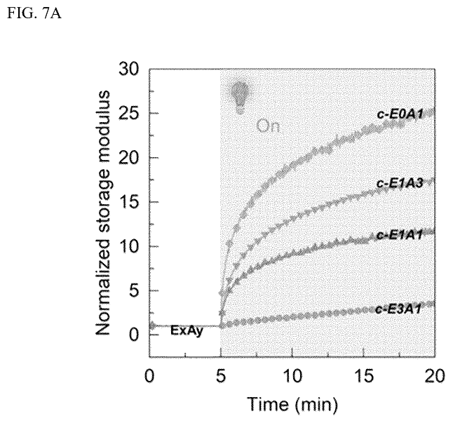

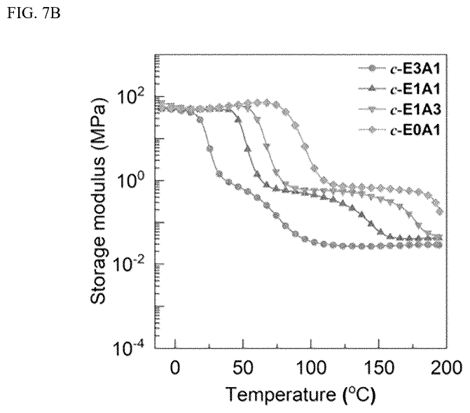

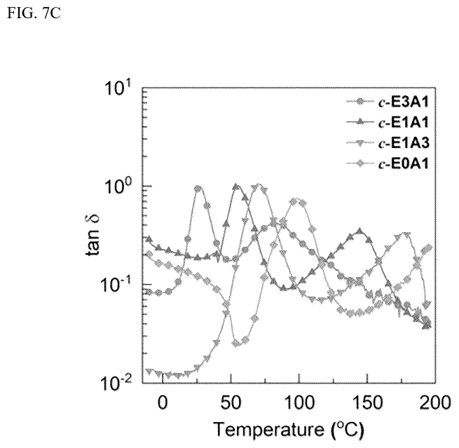

FIGS. 7A to 7C are graphs illustrating the changes in storage modulus and tan according to the temperature from the difference in copolymerization ratio of chemically crosslinked polymer networks composed of the polymer material prepared according to the embodiment. Since the polymer material according to Example 1 is a material in which the supramolecular polymer network can be transformed into a chemically crosslinked polymer network as in Example 2 under long-wavelength ultraviolet light, the accompanying changes in thermal and mechanical properties were confirmed.

Referring to FIG. 7A, according to the content of anthracene, the chemically crosslinked polymer networks exhibited a storage modulus that increased by at least 3.5-fold and as much as 25-fold. In addition, referring to FIG. 7B, it could be confirmed that a second glass transition temperature appeared in addition to the glass transition temperature of the existing network. Furthermore, it could be confirmed that the temperature difference between the first and second glass transition temperatures becomes wider according to the content of anthracene. Even in FIG. 7C, unlike FIG. 5C, it can be confirmed that a peak exhibiting the highest tan δ value appears twice, and therefrom, it could be confirmed that two glass transition temperatures appear in the chemically crosslinked polymer network.

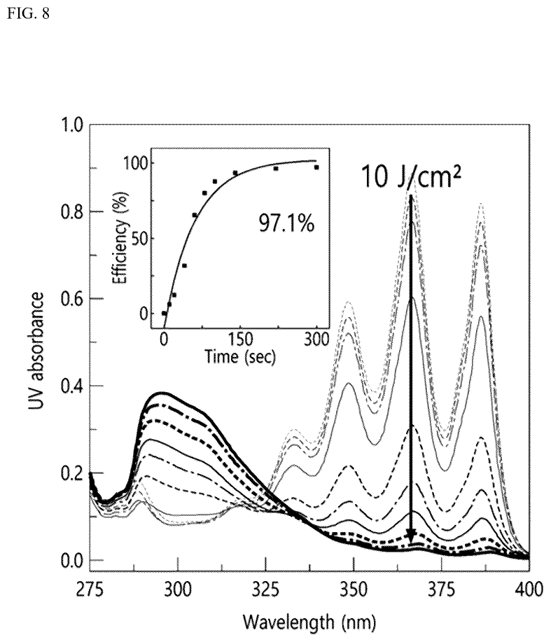

FIG. 8 is a graph for confirming the photodimerization efficiency of the chemically crosslinked polymer network prepared according to the embodiment. Considering that the intrinsic UV absorption characteristics of molecules disappear when a photodimerization reaction between anthracene molecules is performed, it was observed in a solution over the UV irradiation time, using E0A1 as a representative, whether the changes in the optical absorption characteristics of the polymer appeared.

Referring to FIG. 8, it could be confirmed that the chemically crosslinked polymer network exhibits a high photodimerization efficiency of about 97% in about 5 minutes when irradiated with ultraviolet light using a single long-wavelength (365 nm) ultraviolet lamp with an intensity of about 33 mW/cm².

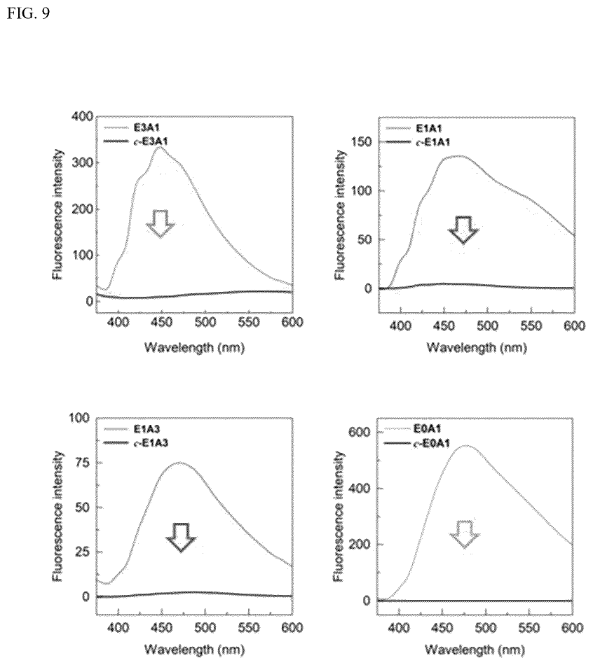

FIG. 9 is a graph for confirming the photodimerization efficiency of chemically crosslinked polymer networks having different copolymerization ratios, prepared according to the embodiment.

Considering that when the photodimerization reaction between anthracene molecules is performed, the molecular conjugation structure is broken and the previously observed fluorescent emission characteristics are lost, it was evaluated to what degree photodimerization efficiency in a solid phase all the ExAys including anthracene have.

Referring to FIG. 9, it was calculated from the fluorescence emission intensity that all polymers showed a dimer conversion rate of 90% or more, and based on this, it could be confirmed that a chemically crosslinked supramolecular polymer network was formed without any problems.

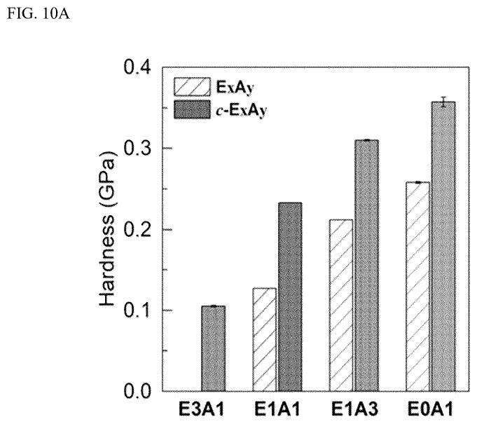

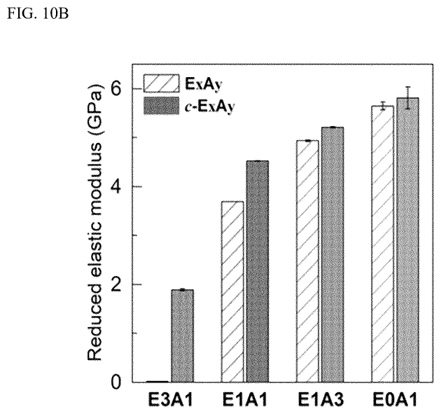

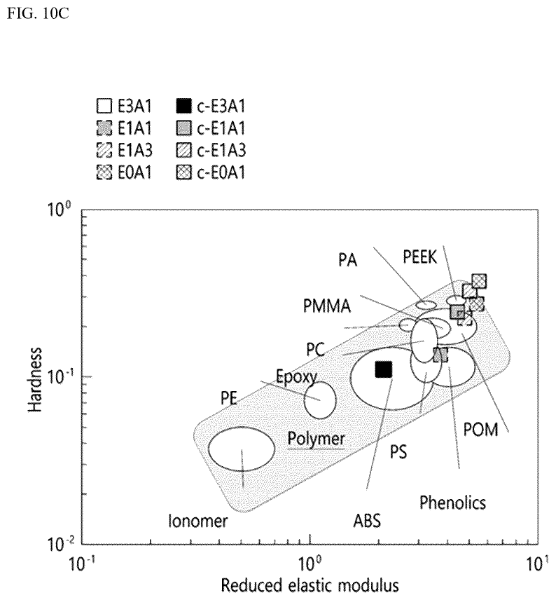

FIGS. 10A to 10C are graphs for grasping hardness and mechanical properties of polymer materials and chemically crosslinked polymer networks having different copolymerization ratios, prepared according to the embodiment. In order to determine whether the polymer materials synthesized by the examples in the present invention have mechanical properties suitable for use as protective coating films, an indentation test was performed.

Referring to FIGS. 10A and 10B, it could be confirmed that as the content of anthracene increases, the hardness and reduced elastic modulus have higher values. In addition, it could be confirmed that the hardness and storage modulus values increased as the supramolecular polymer network (ExAy) was converted into a chemically crosslinked polymer network (c-ExAy). Referring to FIG. 10C corresponding to the Ashby plot, it could be confirmed that among the chemically crosslinked polymer networks, c-E1A1, c-E1A3, and c-E0A1 have excellent mechanical properties compared to other plastic materials.

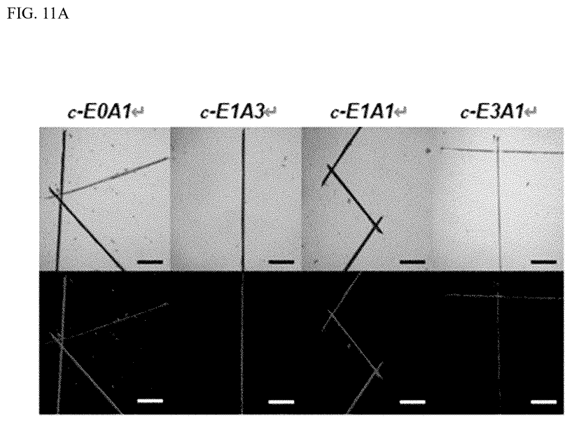

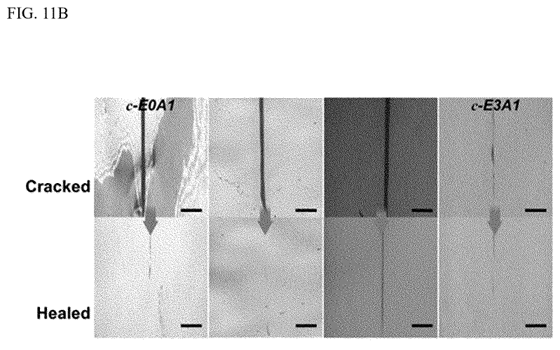

FIGS. 11A and 11B are photographs illustrating the self-healing ability of polymer materials having different copolymerization ratios prepared according to the embodiment according to the external stimulus.

Referring to FIG. 11A, when a chemically crosslinked polymer network (c-ExAy) coated on a substrate was damaged using a razor, it could be confirmed that fluorescence was clearly emitted from all the polymers according to the damaged site.

Further, photodimerized molecules may be restored to their original molecular structure by breaking the covalent bonds using heat. Referring to FIG. 11B, it could be confirmed that as a result of heating the chemically crosslinked polymer network (c-ExAy) to about 130° C. or more, and then again irradiating the polymer network with ultraviolet light, the damaged sites were self-healed. In this case, it could be confirmed that the lower the anthracene content of the supramolecular polymer network, the better its self-healing ability.

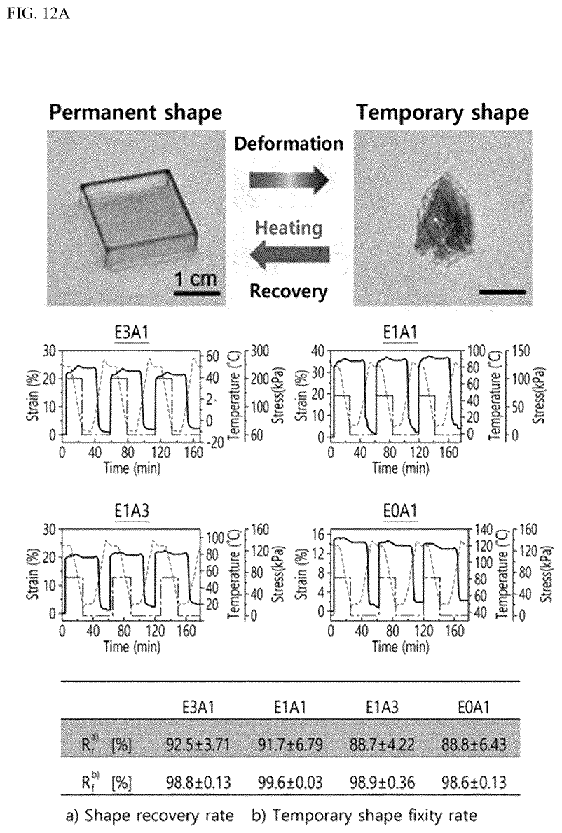

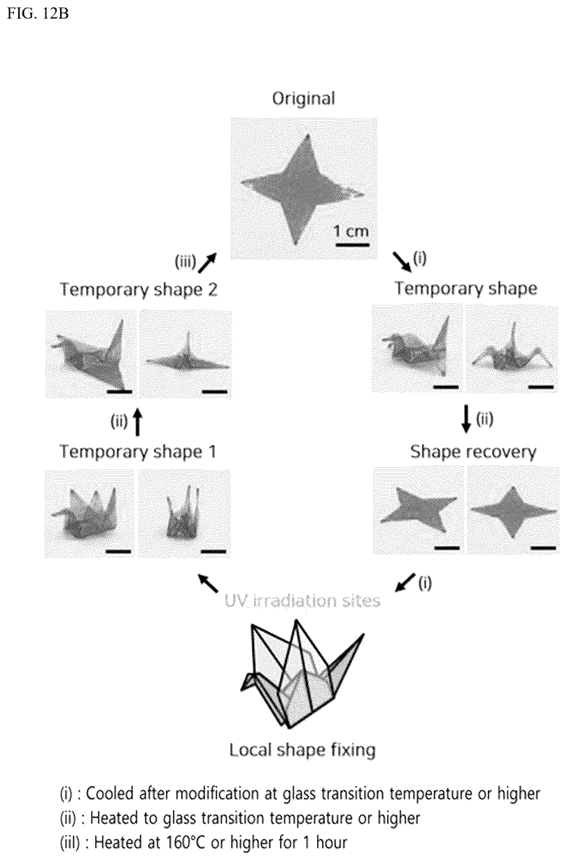

FIGS. 12A and 12B are views illustrating the dual-shape memory properties of the polymer material prepared according to the embodiment. In addition to the damage sensing and self-healing characteristics of the polymer materials prepared by the examples, it was confirmed whether the supramolecular polymer network exhibits excellent dual-shape memory properties.

Referring to FIG. 12A, through a cyclic shape memory test, it could be confirmed that the polymer material containing anthracene exhibited a high temporary shape fixing ability (Rf) of about 98% or more, and the polymer material had a high shape recovery ability (Rr) of about 90% or more.

Additionally, it was confirmed that, in addition to its existing shape-memory properties, E1A1 among the polymer materials can be fixed in a new form by locally irradiating E1A1 with ultraviolet light to elicit enhanced thermal and mechanical properties. Referring to FIG. 12B, it could be confirmed that when E1A1 is heated to a temperature equal to or higher than the glass transition temperature (ii), a site composed only of the supramolecular polymer network is restored to its existing shape to implement a new temporary shape. Furthermore, as a result of applying heat to the chemically crosslinked polymer network (iii), it could be confirmed that the supramolecular polymer network can return to its existing shape by restoring the polymer network to its original structure.

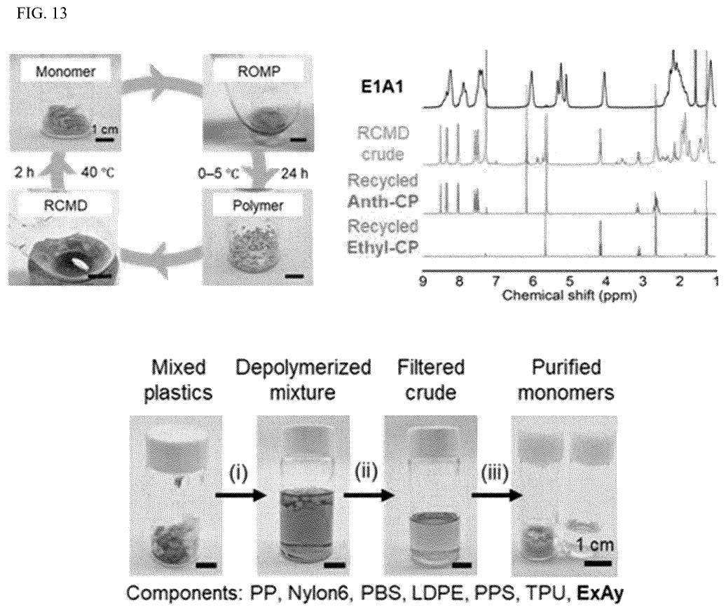

FIG. 13 is a view illustrating a process of recycling the polymer material prepared according to the embodiment through depolymerization.

Referring to FIG. 13, it could be confirmed that the polymer material prepared by the example was depolymerized and restored to monomers within 2 hours at a relatively mild temperature of 40° C. in the presence of the same catalyst as that used during the polymerization. The initially swollen polymer networks were completely dissolved after depolymerization, and it could be confirmed through NMR analysis that the polymer networks were converted into monomers with a purity of 99% or more. Thereafter, the monomer that constitutes the polymer material was isolated and recovered through column purification. Through this, it could be confirmed that the present polymer material can be selectively applied to the recycling process even though present in a mixed state with most general-purpose polymers that do not have carbon-carbon double bonds in the polymer chain.

The above-described content is merely illustrative, and various modifications may be made by a person having ordinary skill in the art to which the present invention pertains without departing from the scope and technical spirit of the described embodiments. The above-described embodiments may be implemented individually or in any combination.

### INDUSTRIAL APPLICABILITY

The present invention can be used in related fields in which protective coating materials are required, such as displays, optical instruments, and vehicles.

1. A novel monomer formed by a bond between the following Chemical Formula 1 and any one of the following Chemical Formulae 2 to 4: 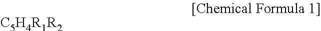
here, Chemical Formula 1 is a ring type, R₁ is a hydroxyl group, a carboxyl group, an amine group, chlorine (CI), bromine (Br) or iodine (I), and R₂ is a hydroxyl group or hydrogen; 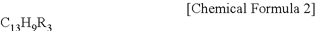
here, R₃ is a carboxyl group, an isocyanate group, a halogen group, a halide methyl group, an amine group, a hydroxymethyl group, a boronic acid group, an aldehyde group, a phenylmethanimine group, or an ethylene group, and the halogen element is chlorine, bromine, or iodine; 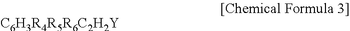
here, R₄ is hydrogen, a methoxy group, an acetoxy group, a trifluoromethyl group, a methylseleno group, a hydroxyl group, bromine, chlorine or an amine group, R₅ is a hydroxyl group or hydrogen, R₆ is a methoxy group or hydrogen, and Y is a carboxyl group, an acyl chloride group, or chlorine; and 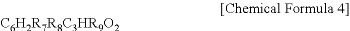
here, R₇ is a hydroxyl group, an amine group, bromine, or chlorine, R₈ is hydrogen, a methoxy group, an acetyl group, a methyl group, a hydroxyl group, or chlorine, and R₉ is a trifluoromethyl group, a methyl group, or hydrogen.

2. The novel monomer of claim 1, wherein Chemical Formula 1 is represented by any one of the following Chemical Formulae 1-1 to 1-5:

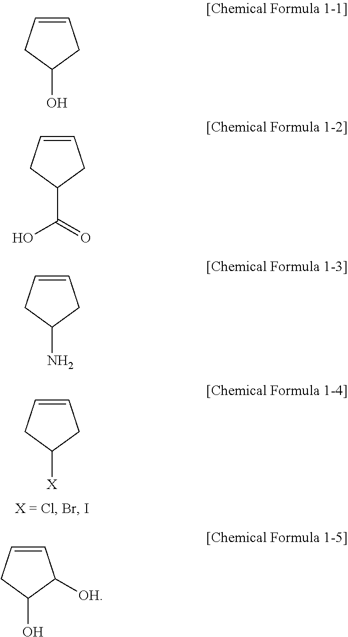

3. The novel monomer of claim 1, wherein Chemical Formula 2 is represented by any one of the following Chemical Formulae 2-1 to 2-10:

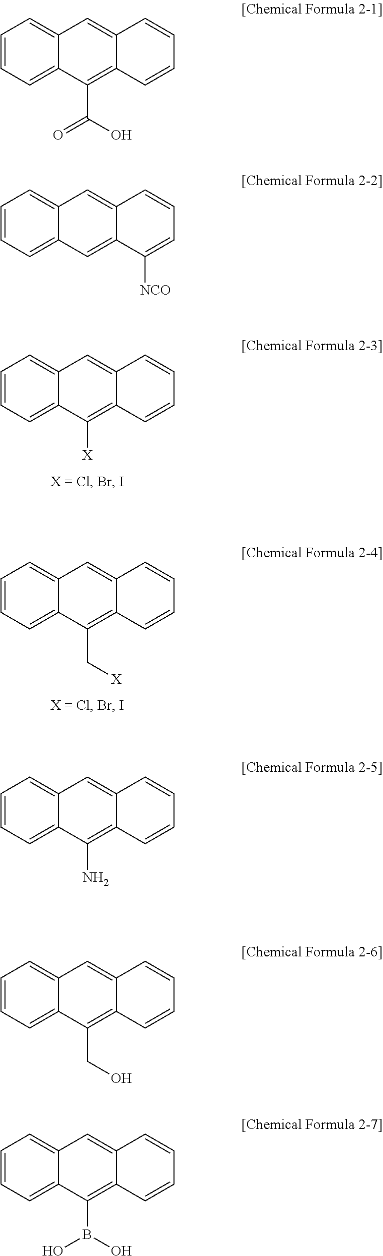

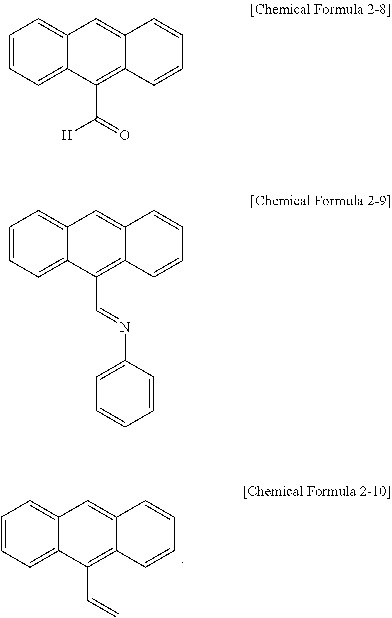

4. The novel monomer of claim 1, wherein Chemical Formula 3 is represented by any one of the following Chemical Formulae 3-1 to 3-14:

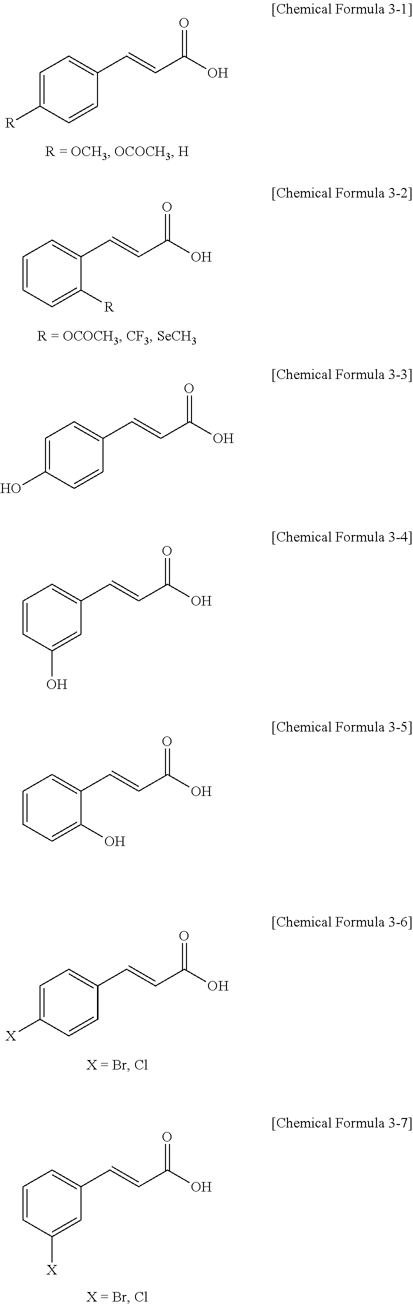

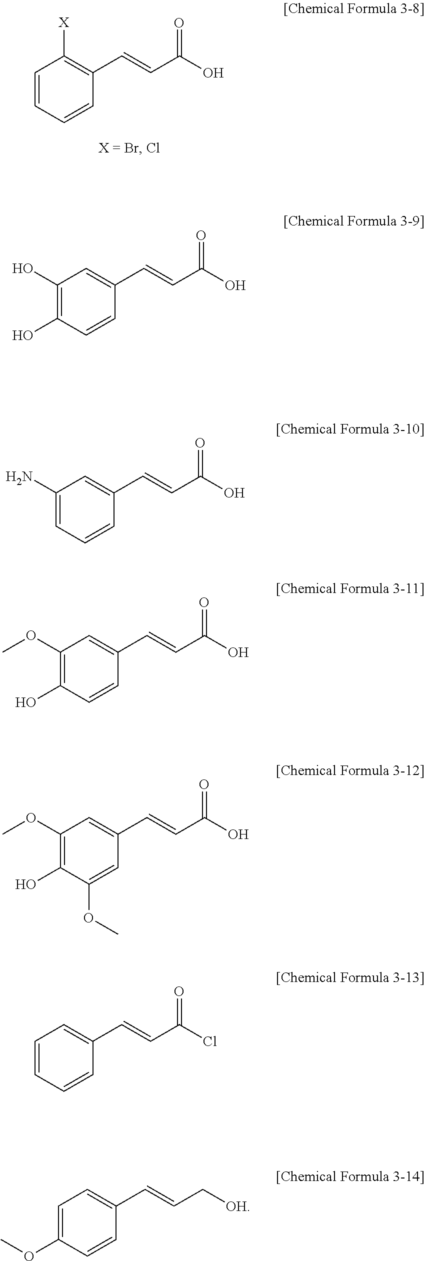

5. The novel monomer of claim 1, wherein Chemical Formula 4 is represented by any one of the following Chemical Formulae 4-1 to 4-11:

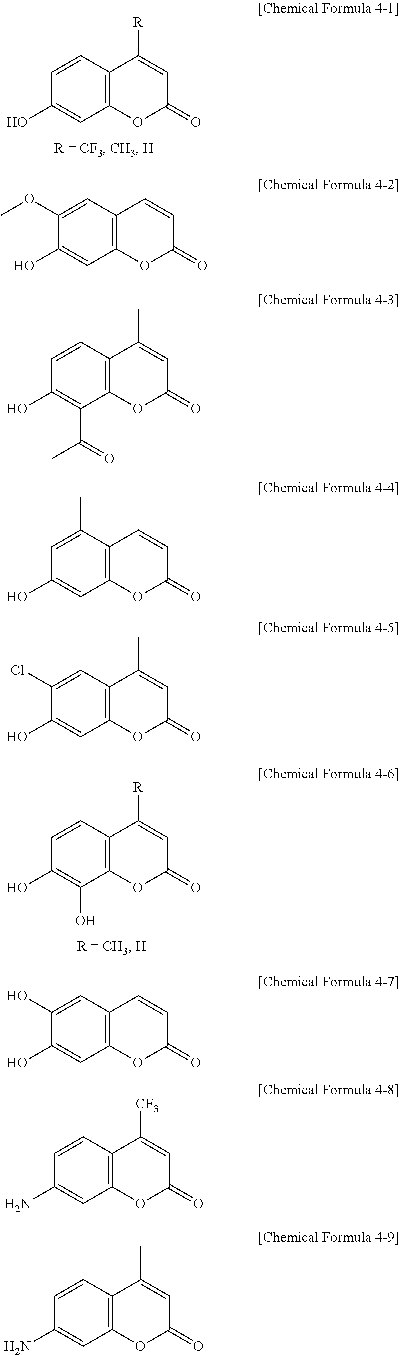

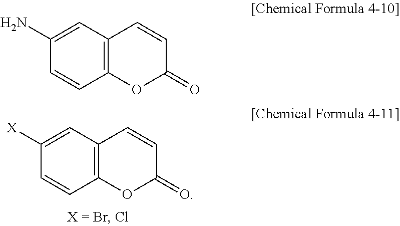

6. The novel monomer of claim 2, wherein the novel monomer is represented by any one of the following Chemical Formulae 5-1 to 5-20:

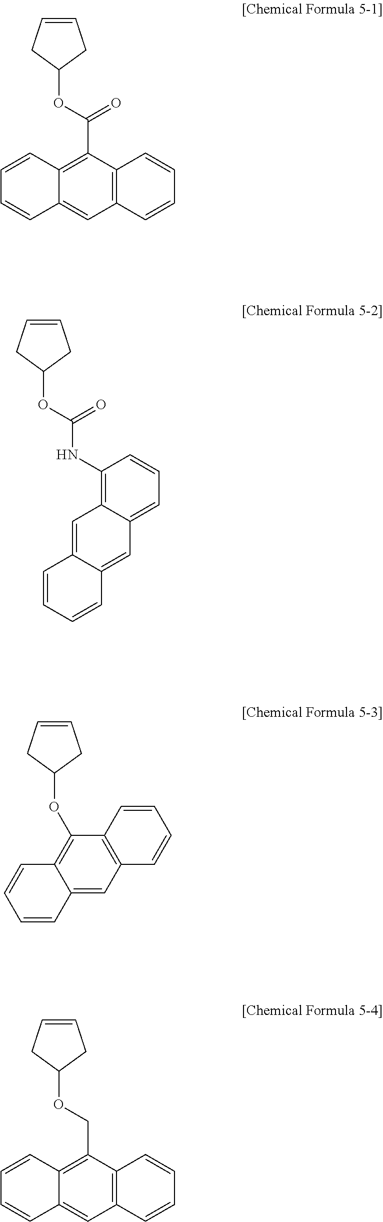 [SMILES: O=C(OC1CC=CC1)c1c2ccccc2cc2ccccc12 ; O=C(Nc1cccc2cc3ccccc3cc12)OC1CC=CC1 ; C1=CCC(Oc2c3ccccc3cc3ccccc23)C1 ; C1=CCC(OCc2c3ccccc3cc3ccccc23)C1]

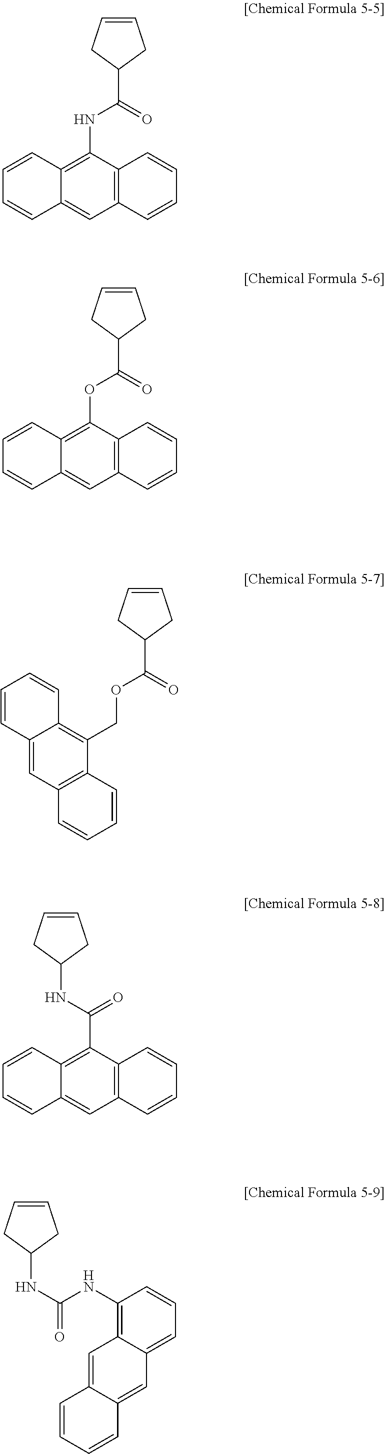 [SMILES: O=C(Nc1c2ccccc2cc2ccccc12)C1CC=CC1 ; O=C(Oc1c2ccccc2cc2ccccc12)C1CC=CC1 ; O=C(OCc1c2ccccc2cc2ccccc12)C1CC=CC1 ; O=C(NC1CC=CC1)c1c2ccccc2cc2ccccc12 ; O=C(Nc1cccc2cc3ccccc3cc12)NC1CC=CC1]

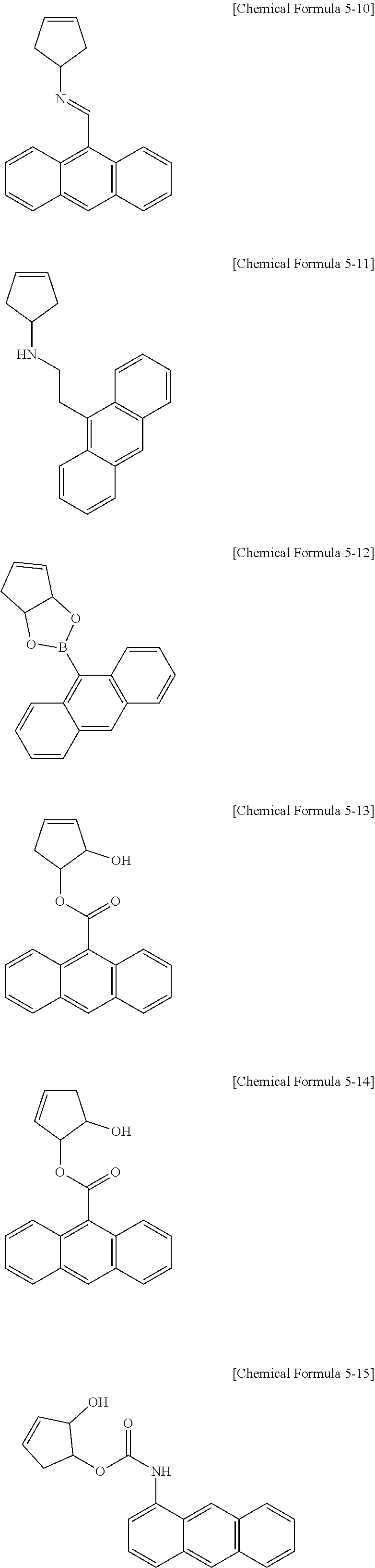 [SMILES: C1=CCC(/N=C/c2c3ccccc3cc3ccccc23)C1 ; C1=CCC(NCCc2c3ccccc3cc3ccccc23)C1 ; C1=CC2OB(c3c4ccccc4cc4ccccc34)OC2C1 ; O=C(OC1CC=CC1O)c1c2ccccc2cc2ccccc12 ; O=C(OC1C=CCC1O)c1c2ccccc2cc2ccccc12 ; O=C(Nc1cccc2cc3ccccc3cc12)OC1CC=CC1O]

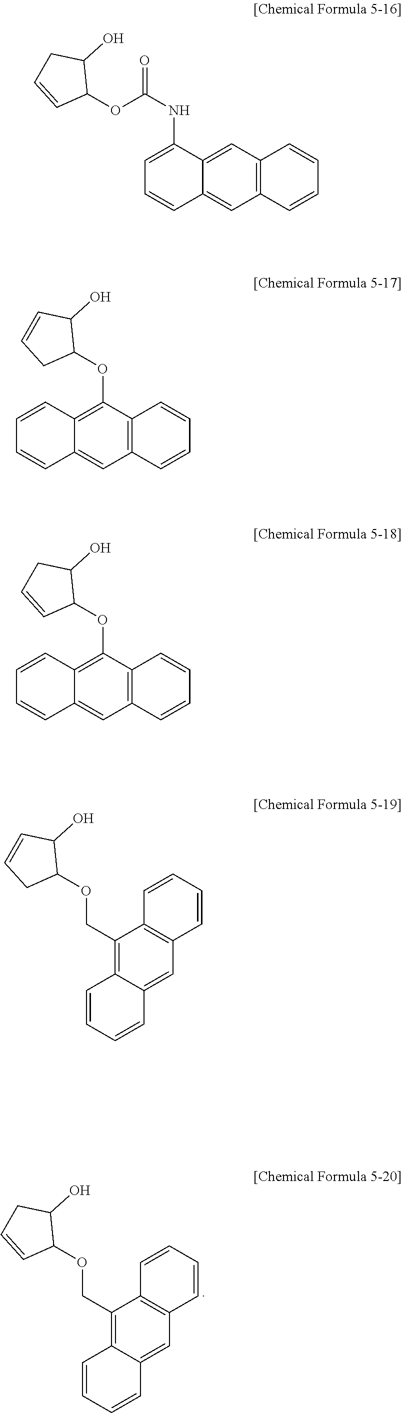 [SMILES: O=C(Nc1cccc2cc3ccccc3cc12)OC1C=CCC1O ; OC1C=CCC1Oc1c2ccccc2cc2ccccc12 ; OC1CC=CC1Oc1c2ccccc2cc2ccccc12 ; OC1C=CCC1OCc1c2ccccc2cc2ccccc12 ; OC1CC=CC1OCc1c2ccccc2cc2ccccc12]

7. The novel monomer of claim 2, wherein the novel monomer is represented by any one of the following Chemical Formulae 6-1 to 6-65:

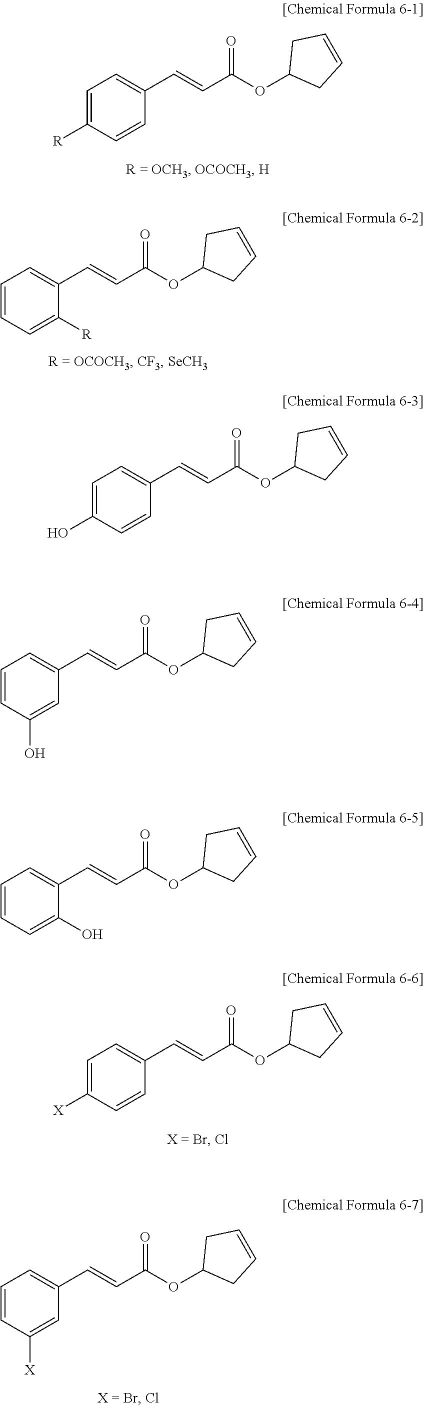

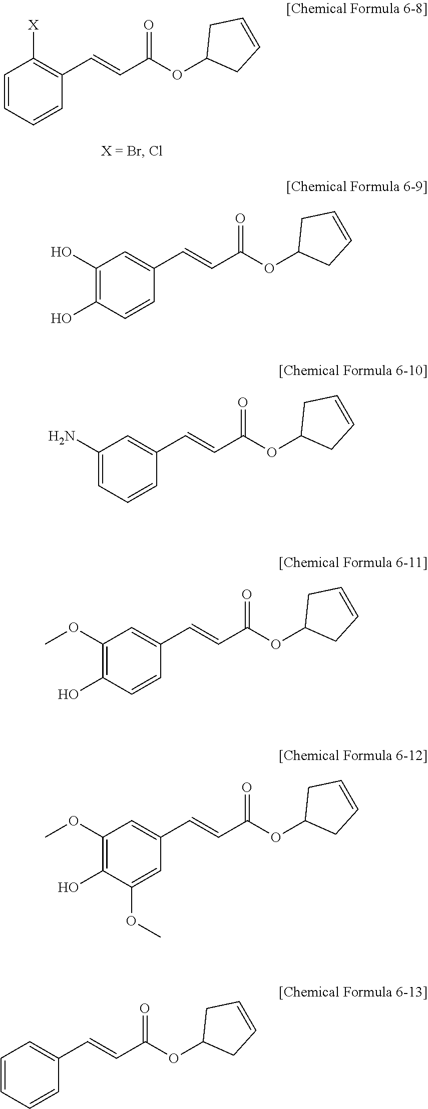

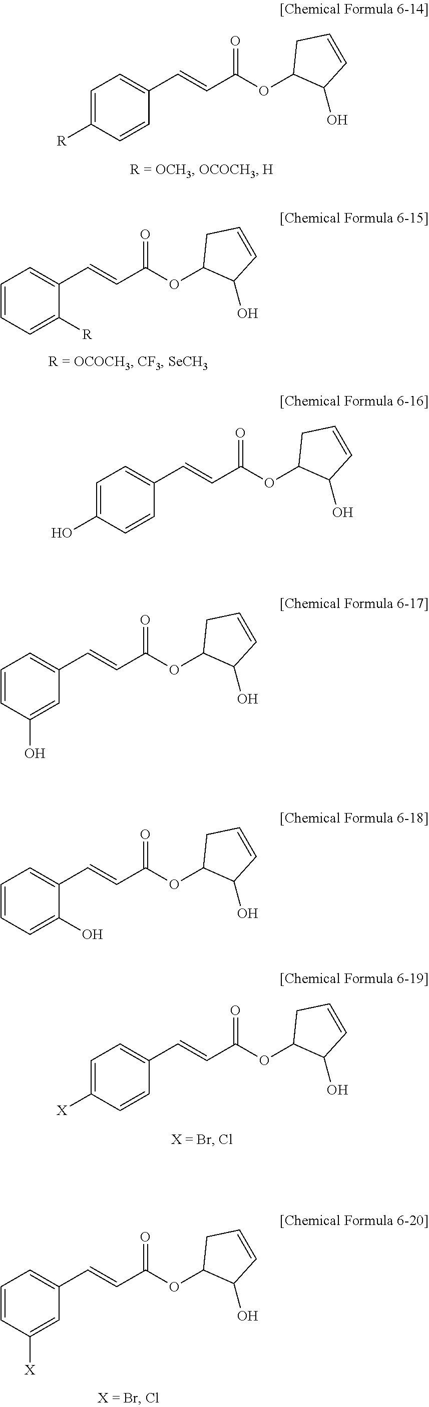

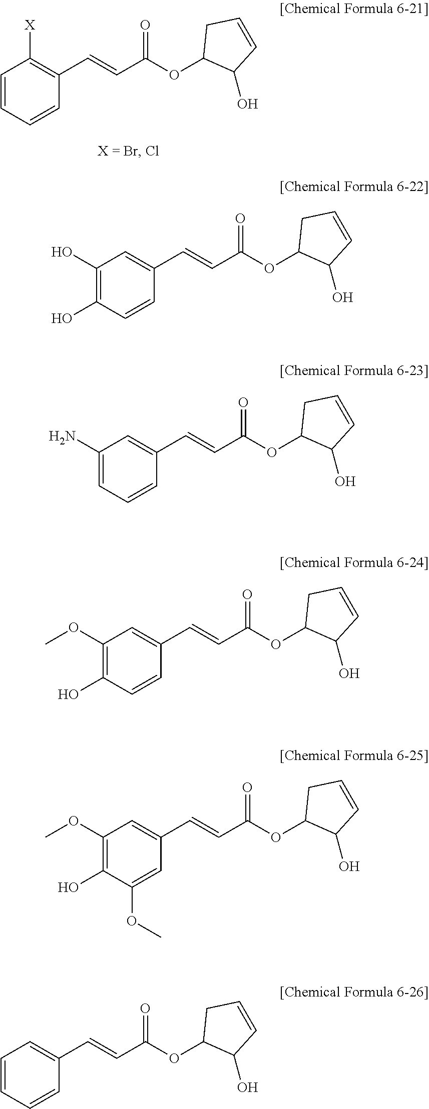

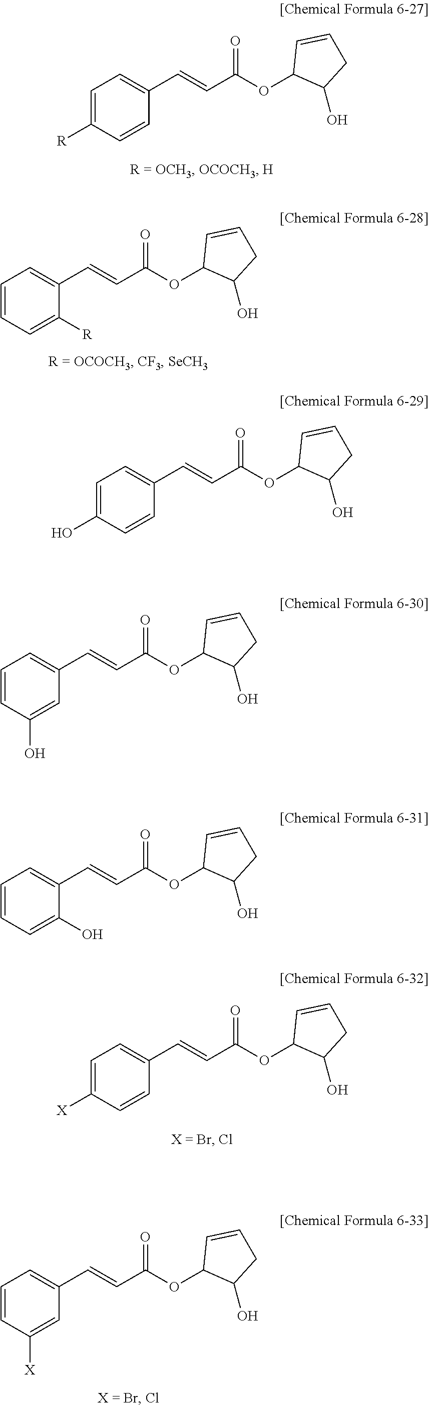

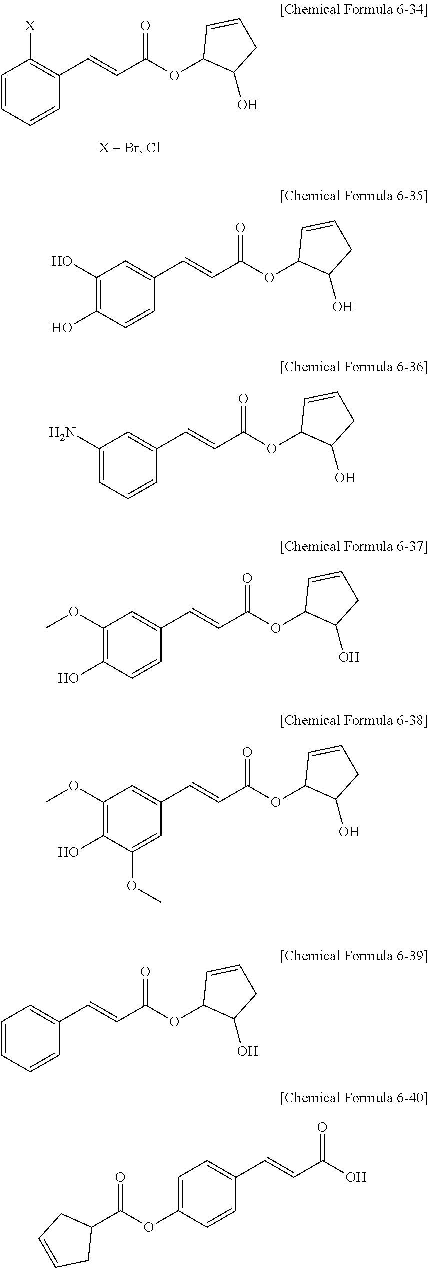

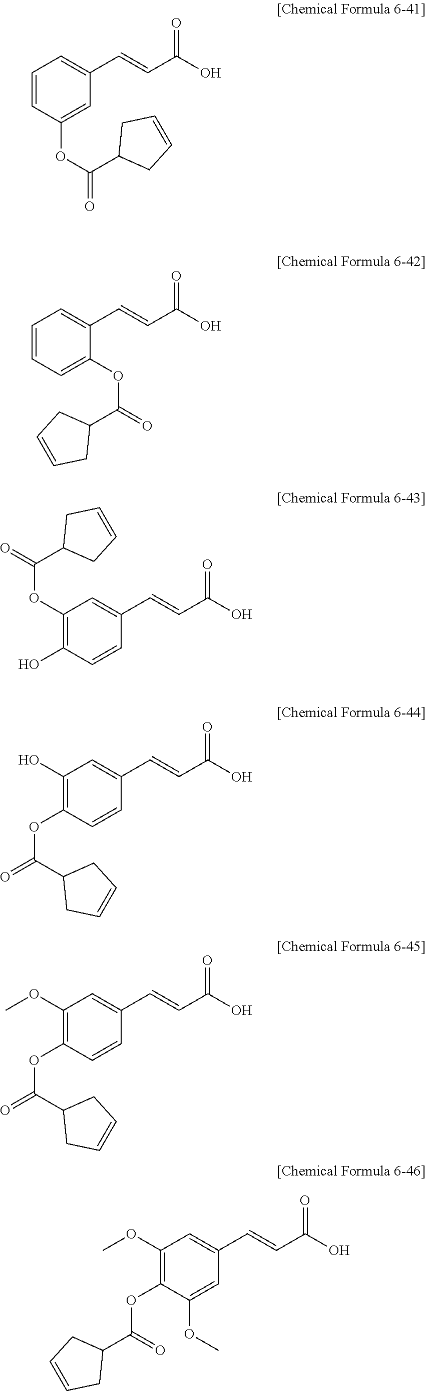 [SMILES: O=C(O)/C=C/c1cccc(OC(=O)C2CC=CC2)c1 ; O=C(O)/C=C/c1ccccc1OC(=O)C1CC=CC1 ; O=C(O)/C=C/c1ccc(O)c(OC(=O)C2CC=CC2)c1 ; O=C(O)/C=C/c1ccc(OC(=O)C2CC=CC2)c(O)c1 ; COc1cc(/C=C/C(=O)O)ccc1OC(=O)C1CC=CC1 ; COc1cc(/C=C/C(=O)O)cc(OC)c1OC(=O)C1CC=CC1]

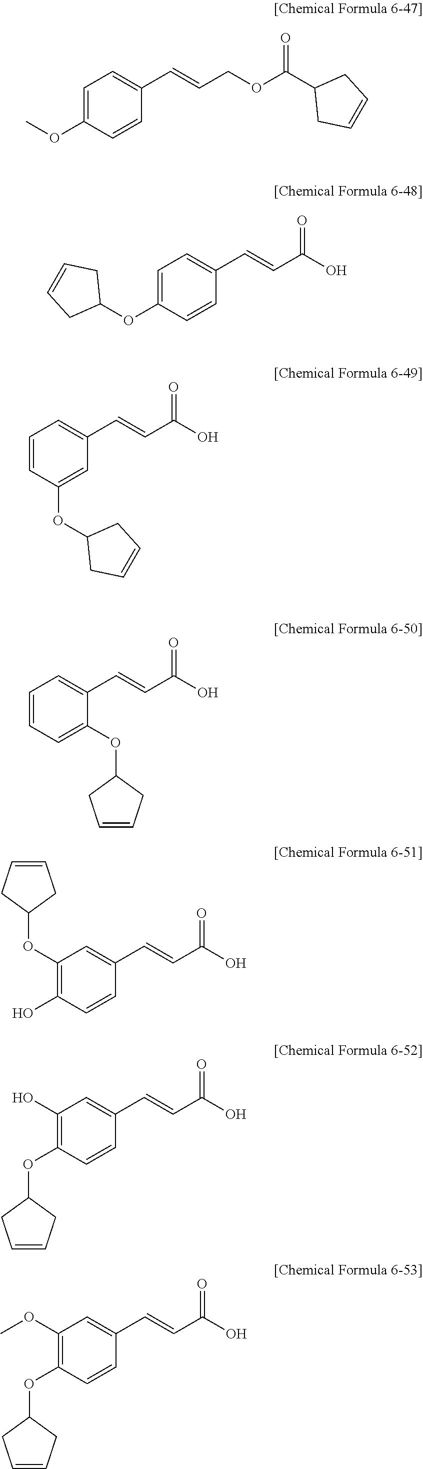

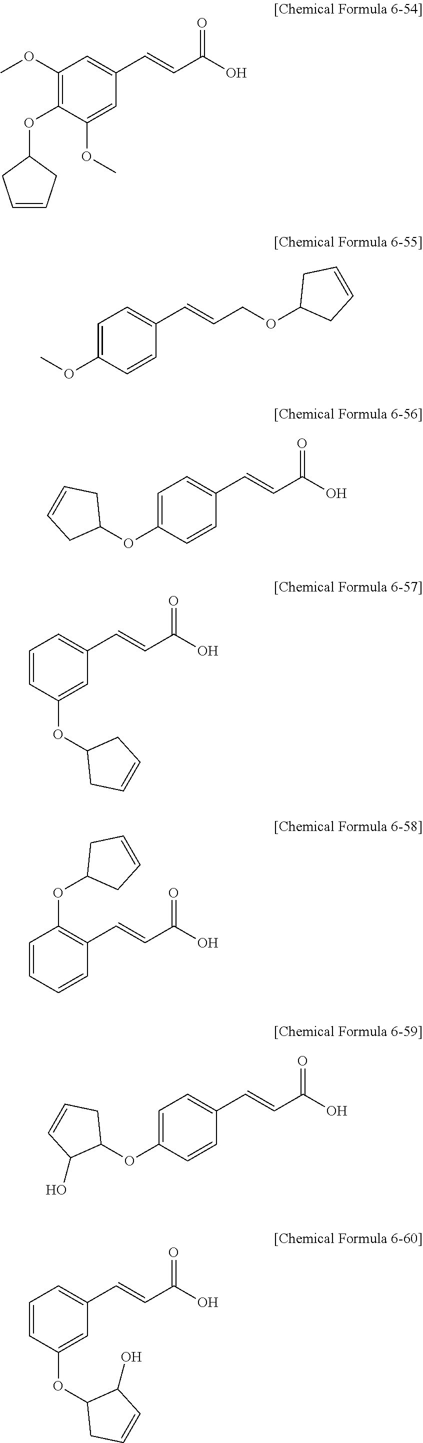

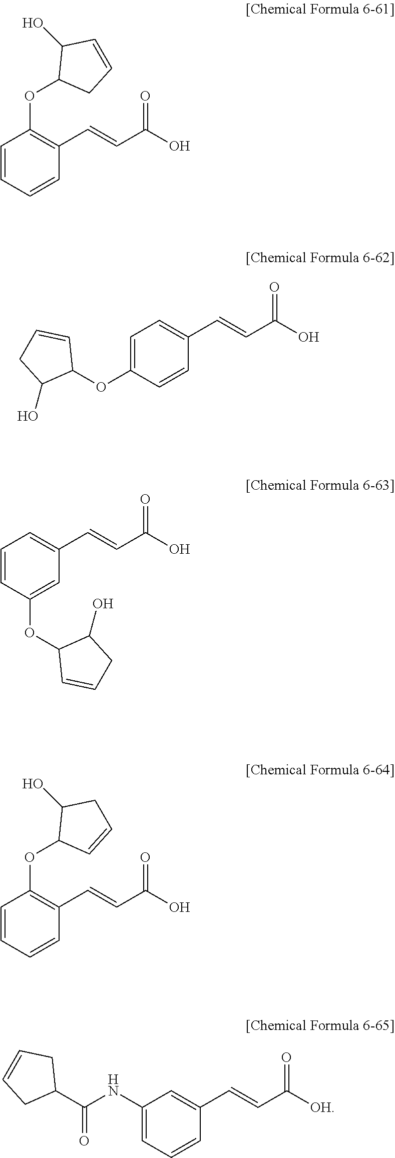 [SMILES: O=C(O)/C=C/c1ccccc1OC1CC=CC1O ; O=C(O)/C=C/c1ccc(OC2C=CCC2O)cc1 ; O=C(O)/C=C/c1cccc(OC2C=CCC2O)c1 ; O=C(O)/C=C/c1ccccc1OC1C=CCC1O ; O=C(O)/C=C/c1cccc(NC(=O)C2CC=CC2)c1]

8. The novel monomer of claim 2, wherein the novel monomer is represented by any one of the following Chemical Formulae 7-1 to 7-27:

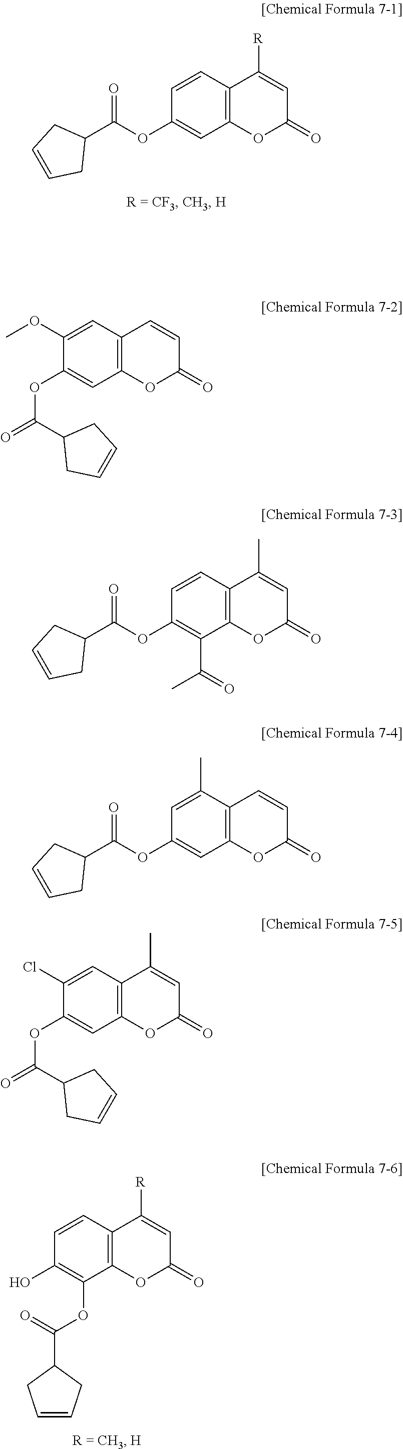

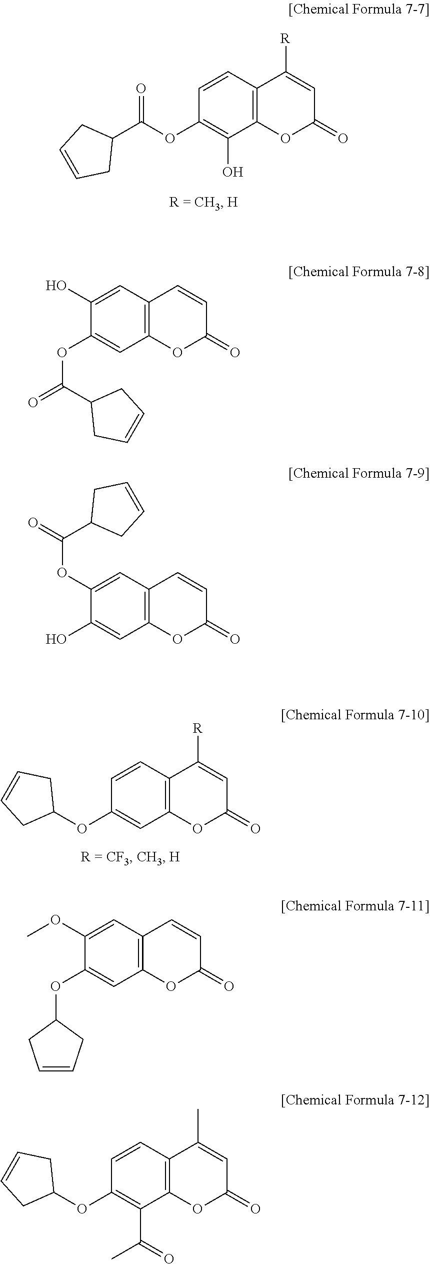

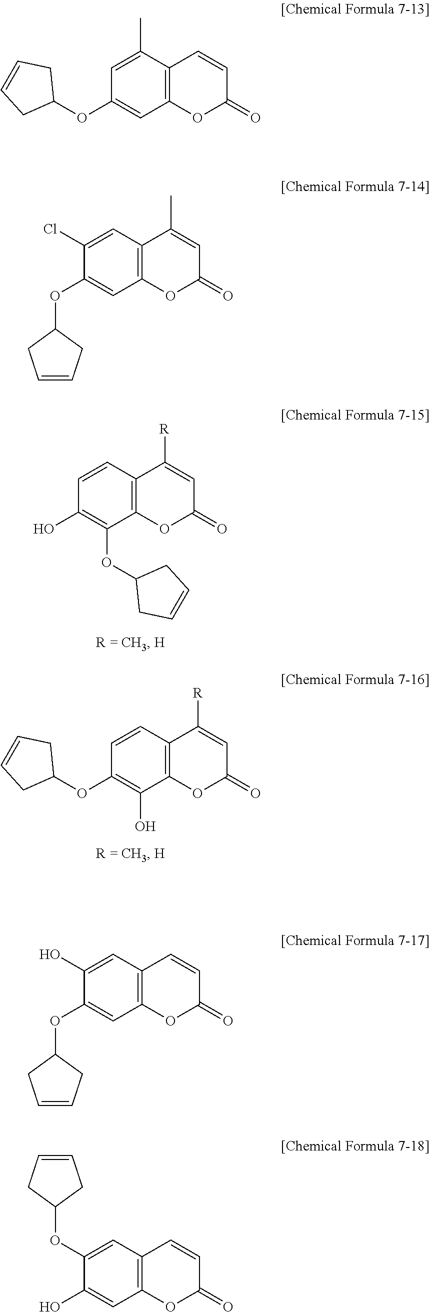

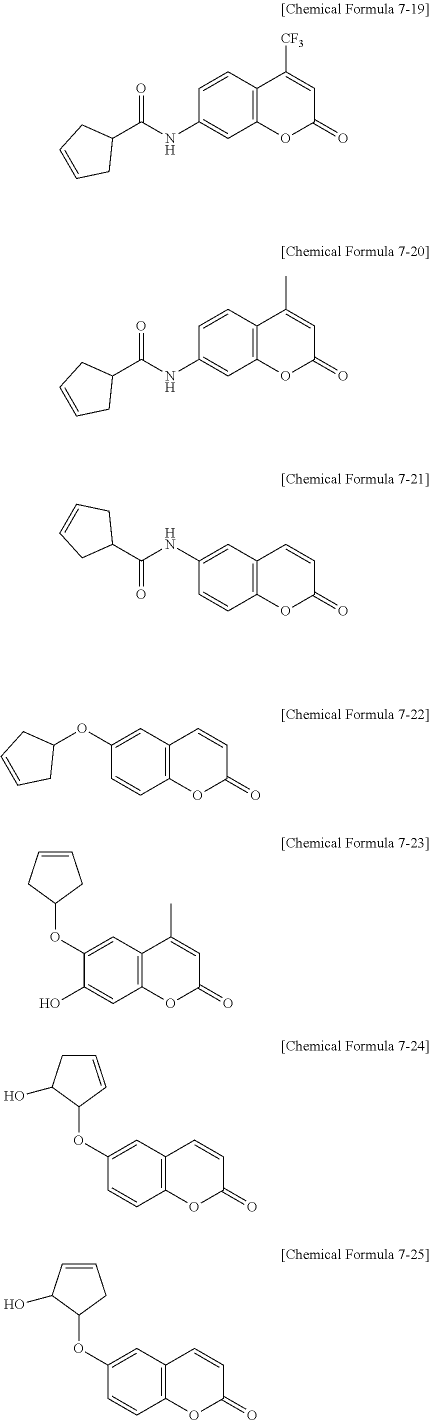

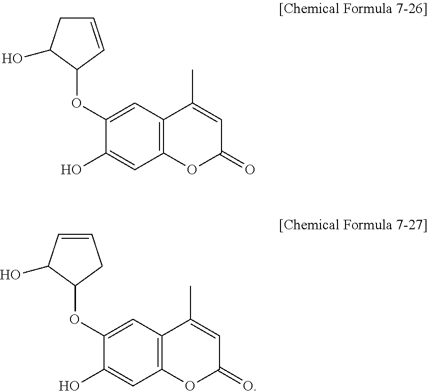 [SMILES: Cc1cc(=O)oc2cc(O)c(OC3C=CCC3O)cc12 ; Cc1cc(=O)oc2cc(O)c(OC3CC=CC3O)cc12]

9. The novel monomer of claim 6, wherein the novel monomer has a ring strain of more than 2 kcal/mol and less than 8 kcal/mol.

10. A polymer material capable of maintaining a chemical nature, comprising the novel monomer described in claim 1.

11. The polymer material capable of maintaining a chemical nature of claim 10, wherein a molar ratio of the novel monomer in the polymer material is 1 mol % or more.
